(19) United States
(12) Patent Application Publication
(10) Pub. No.: US 2019/0148019 A1
(43) Pub. Date: May 16, 2019

(54) SYSTEM FOR PREDICTING EFFICACY OF A TARGET-DIRECTED DRUG TO TREAT A DISEASE

(71) Applicant: HOFFMANN-LA ROCHE INC., Little Falls, NJ (US)

(72) Inventors: Markus BUNDSCHUS, Muenchen (DE); Fabian HEINEMANN, Muenchen (DE); Christian MEISEL, Hohenschaeftlarn (DE); Torsten HUBER, Berlin (DE); Ulf LESER, Berlin (DE)

(73) Assignee: HOFFMANN-LA ROCHE INC., Little Falls, NJ (US)

(21) Appl. No.: 16/300,371

(22) PCT Filed: May 5, 2017
(86) PCT No.: PCT/EP2017/060844

§ 371 Date: Nov. 9, 2018

(30) Foreign Application Priority Data
May 12, 2016 (EP) 16169452.6

(51) Int. Cl. G16H 50/20; G16H 20/10; G06N 20/00; A61B 5/00

(52) U.S. Cl. CPC G16H 50/20; G16H 20/10; A61B 5/7264; A61B 5/4848; G06N 20/00

(57) ABSTRACT

The system includes a processor configured for receiving biomedical documents including an identifier of the target and/or of the disease; specifying an offset time the offset time indicating a time interval ahead of the performing of the prediction; specifying a time window ending at the begin of the offset time; extracting a plurality of features selectively from the ones of the received documents published during the time window; providing a classifier having been trained on training features extracted from biomedical training documents published within a training time window ending at the begin of the offset time ahead of a moment the outcome of one or more training studies on training target-disease-pairs was disclosed; executing the classifier, thereby providing the extracted features as input; and outputting a classification result indicating whether the drug directed at the target can be used to treat the disease.

### FIELD OF THE INVENTION

The invention relates to the field of machine-learning, and more particularly to the field of predicting the efficacy of a drug to treat a disease.

### BACKGROUND AND RELATED ART

The development of drugs is time-consuming and expensive. Failures in clinical trials, in particular late-stage clinical trials, are a major cost driver for pharmaceutical companies. Methods which provide some insights on the success chances of new potential drugs may therefore be of great help for deciding if further resources should be spent on the development and clinical testing of a particular drug.

Previous work has been performed, for instance, on using text mining approaches for detecting new ‘game-changing’ technology areas (Reardon, S. 2014: “Text-mining offers clues to success”, Nature 509, 1). Moreover, it has been reported that a high number of publications may indicate towards the success of such a drug in a clinical trial (Joshi, V. and Milletti, F., 2014, “Quantifying the probability of clinical trial success from scientific articles”, Drug discovery today 19 (10), 1514-1517). However, the current tools and technologies are not able to accurately predict the outcome of a clinical trial. In the article “A Tool for Predicting Regulatory Approval After Phase II Testing of New Oncology Compounds”, ICAL PHARMACOLOGY & THERAPEUTICS, VOLUME 98 NUMBER 5, November 2015, JA DiMasi et al. describe an algorithm for predicting regulatory marketing approval for new cancer drugs after phase II testing. Data on safety, efficacy, operational, market, and company characteristics were obtained from public sources and logistic regression and machine-learning methods were used to assess overall predictability.

### SUMMARY

It is an objective of the present invention to provide for an improved method, system and computer readable storage medium for predicting the outcome of a medical study as specified in the independent claims. Embodiments of the invention are given in the dependent claims. Embodiments of the present invention can be freely combined with each other if they are not mutually exclusive.

In one aspect, the invention relates to a method for predicting an outcome of a medical study. The medical study evaluates the efficacy of a drug directed at a target to treat a disease. The method is implemented in an electronic system and comprises:
receiving biomedical documents comprising an identifier of the target or of the disease or of the target and the disease;
specifying an offset time, the offset time indicating a time interval ahead of the performing of the prediction;
specifying a time window of predefined duration, the window ending at the begin of the offset time;
extracting a plurality of features selectively from the ones of the received documents published during said time window;
providing a classifier having been trained on a set of training features extracted from a set of biomedical training documents, the training documents published within a training time window ending at the begin of the offset time ahead of a moment the outcome of one or more training studies on training target-disease-pairs was disclosed;
performing the prediction by executing the classifier, thereby providing the extracted features as input to the classifier;
outputting a result of the classifier, the result predicting the efficacy of the drug directed at the target to treat the disease.

Using an offset time for defining the boundaries of a time window used for selecting documents to form the data source of feature extraction and to provide the extracted features as input to a classifier trained on training features whose extraction is based on the same offset time may be advantageous as it has been observed that considering also the offset time ahead of the day of publishing the outcome of a study may significantly increase the prediction accuracy. At the moment of performing literature-based prediction regarding the outcome of a current medical study, not only the outcome of the study is unclear, but also the time when the study will provide a statistically significant result regarding the efficacy of the drug directed at a target to treat a disease. So it is not clear at the moment of performing the prediction when enough biomedical data will have been collected to clearly determine if the drug directed at said target is effective or not. The question if a drug directed at a particular target is effective for treating a particular disease implicitly also reveals if the particular target is a molecule having a biochemical activity whose modification is capable of treating said disease.

Using a time window having a fixed size may further have the advantage that the training procedure of the classifiers is always the same irrespective of the number of years having lapsed since the first mentioning of a drug-disease pair and the outcome of a study was disclosed. Thus, the same type of (untrained) classifier may be trained on training data sets comprising target-disease pairs which cover very different time intervals since a first co-mentioning in a document (spanning e.g. from 5-6 years up to 30 years or longer). No reconfiguration of the un-trained classifier before starting the training phase may be needed.

Extracting the features only from documents published during the time window—and not simply from all documents available/having been published prior to the prediction—may not result in a reduction or even in an increase of the accuracy of the prediction. This is a surprising observation: in general, as many input data as possible is gathered for feeding machine learning based classifiers in order to enlarge the data basis of the decision and thereby also the accuracy of the decision. In contrast to this general approach commonly used in the field of machine learning, only a defined subset of the available documents (the documents published during the time window or parts thereof, but not published during the offset time or published prior to the start of the time window) is used for extracting the features. Moreover, a classifier was used that was also trained only on a defined subset of the available documents. Nevertheless, it has been observed that taking into consideration also the time distance of a prediction time relative to a disclosure time (disclosure of an outcome of a study, the outcome indicating if a drug directed at a particular target is capable of treating a disease) may compensate and even over-compensate the accuracy loss typically involved with a reduction of the size of the data basis.

Extracting features selectively for a time window ending at a defined offset time and feeding the extracted features into a machine learning classifier may allow predicting the approval or failure of a targeted cancer drug significantly better than educated guessing.

Embodiments of the invention may allow successfully discriminating drugs directed at a particular target capable of treating a disease from those that are almost successful, i.e., fail only in phase 2/3 clinical studies. Moreover, embodiments of the invention may allow successfully discriminating drugs directed at a particular target capable of treating a disease from those drugs directed at target-disease pairs that never reached or haven't reached yet such a late stage in the drug development process.

Embodiments of the invention may allow, by extracting offset time dependent features from literature an early-on distinction between eventually approved and eventually failed targeted anti-cancer drugs. In particular, embodiments of the invention may provide for trained classifiers being capable of predicting success of drugs in phase 2 or 3 with remarkably high accuracy. Embodiments of the invention may allow automatically identifying and systematically analyzing implicit signals created by thousands of scientists during the drug discovery process through scientific publications. Said implicit signals relate to differences in how researchers collectively publish about findings that ultimately lead to approved drugs for defined targets and about those that fail.

Embodiments of the invention are based on the assumption that the efficacy of a drug to treat a particular disease strongly or even predominantly depends on the question whether a modification of the activity (e.g. modification of the transcription or translation level, of the methylation or phosphorylation pattern, of the transport of the target within or across cells, etc.) of a particular target will treat a particular disease or not.

According to embodiments, the training target-disease pairs are chosen such that any target-disease (T-DI) pair for which a drug with known efficacy or known non-efficacy exists is used either as negative or as a positive T-DI pair. This means that said T-DI pair must not be used as a negative training T-DI pair and as a positive training T-DI pair at the same time. In case for a particular T-DI pair two or more drugs with known ability or inability to treat the disease which are directed at the target exist, only one of the drugs and corresponding data is used for training the classifiers. In this case, for each of the two or more drugs, a respective “decision time” is known, i.e. the time of disclosing the outcome of a study that evaluates whether or not said drug is capable of treating the disease or not. Preferentially, only the drug whose efficacy was examined by the one of the studies having the earliest time of disclosure is used in the training process and the time of disclosure of the outcome of the study corresponding to said drug is used as the “decision time” relative to which the offset time for specifying the window and for retrieving the training documents is determined.

For example, for a given T-DI pair comprising a particular disease and a particular target, a first and a second drug are known which both bind to the target and modify the activity of the target. The first drug is known (e.g. due to an FDA approval in March 2012) to be effective in treating the particular disease. The second drug is known (e.g. due to an FDA rejection in August 2012) to be non-effective in treating the disease. In this case, the “decision time” of the first drug precedes the “decision time” of the second drug. Therefore, data and documents relating to the first drug are considered in the training phase and the corresponding T-DI pair is used as a positive training T-DI pair (the one of the two drugs related to the particular T-DI pair having the earliest “decision time” is the first drug which is known to be effective in treating the disease). If the (negative) outcome regarding the second drug would have been published earlier than the outcome regarding the efficacy of the first drug, said T-DI pair would have been used as a negative training T-DI pair.

Said features may avoid or at least reduce the ambiguity in a set of documents retrieved for a particular T-DI pair which may relate to different drugs with different efficacy.

According to some other embodiments, the retrieving of the documents used as the training documents is implemented such that selectively any documents mentioning the disease and/or the target of a particular target-disease pair which in addition mention a particular one of the two or more drugs are retrieved. In this case, also the retrieval of the documents used for predicting the outcome of a study whether a drug directed at a particular target will be effective in treating a disease or not is implemented such that the retrieved documents in addition are required to mention the name of the drug examined. Thus, different (training and test) documents for different drugs relating to the same T-DI pair are retrieved. Retrieving documents comprising a drug-target-Compound co-occurrence may likewise help avoiding or at least reducing the ambiguity in a set of documents retrieved for a particular T-DI pair which may relate to different drugs with different efficacy.

The expression “outcome of a medical study” as used herein is a result of a medical study that is at least indicative of whether a particular drug directed at a particular target is effective for treating a particular disease or not (irrespective of the drug's safety). Accordingly, a drug may be classified as being effective against the disease irrespective of the drug's safety. In this case the positive and negative target-disease training pairs may be selected only in dependence of the proven ability or inability of a drug directed at a particular disease irrespective of its safety.

According to some embodiments, a drug is only predicted to be effective for treating said disease if it is more efficient than an existing “gold standard” for treating said disease and/or if it is equally efficient than said existing gold standard and has less negative side effects on a patient's health (i.e., is safer than the gold standard).

According to some embodiments, a drug is only predicted to be effective for treating said disease if in addition said drug is predicted to be save, i.e., is predicted not to cause negative side effects that outweigh the health-promoting effects of the drug. In this case the positive and negative target-disease training pairs are selected such that the positive target-disease training pairs consist of target disease pairs for which a respective drug is known that was proven to be effective and save and such that the negative target-disease training pairs consist of target disease pairs for which said drug was proven not to be effective in treating the disease and/or was proven not to be save.

According to some embodiments, the medical study is a scientific publication in the field of basic research that proves—based on current scientific standards—whether a particular drug is effective of treating a particular disease or not. According to other embodiments, the medical study is a study that is performed obtaining a drug approval by a regulatory authority, e.g. the food and drug administration “FDA”, whereby the “outcome” of said study is the final decision of the regulatory authority to approve or to deny the use of the drug for treating the disease. For example, in this case the positive training target-disease pairs comprise targets for which an FDA approval for treating a particular disease exists and the negative training target-disease pairs comprise targets for which such an approval was denied due to lack of effectiveness and/or to lack of safety.

According to embodiments, the offset time is one of a plurality of different, predefined offset times. The trained classifier is one of a plurality of classifiers having been trained on training features extracted from biomedical training documents published within a training time window. The training time windows of each of the classifiers end at a different training offset time, i.e., at different time intervals ahead of a moment the outcome of one or more training studies on training target-disease-pairs was disclosed. The method comprises, for each of the predefined offset times:
specifying a further time window of predefined duration, the further window ending at the predefined offset time;
extracting a plurality of features selectively from the ones of the received documents published during said further time window;
providing the extracted plurality of features as input selectively to the one of the plurality of classifiers having been trained on a set of training features extracted from training documents published within a training time window ending at a training offset time that is identical to the predefined offset time;
performing the prediction by executing the classifier to which the features were provided; and
outputting a result of the classifier, the result predicting the efficacy of the drug directed at the target to treat the disease.

By taking into consideration multiple different offset times for extracting, for each of the offset times, a set of features, and by feeding multiple classifiers such that the input features extracted for a given offset time are provided selectively to the one of the multiple classifiers that has been trained on training features generated based on the same (training) offset time, the time between a report in the literature and the decision on the drug's fate may be taken into account. This may increase the accuracy of the prediction (compared e.g. to simply extract features from all documents available for a particular research topic).

According to embodiments, the method comprises combining the results output by the plurality of executed classifiers for generating a combined result. The combined result is indicative of whether the outcome of the medical study (to be performed in offset time in the future starting from the current time of prediction) will be that the drug directed at a particular target is effective in treating the disease.

By combining the prediction result of multiple classifiers having been trained on a respective offset-time dependent training feature set, the accuracy of the prediction may be increased significantly.

For example, the combination of the results may comprise computing the median of the results generated by all trained classifiers. For example, 10 different offsets (1 year, 2 years, . . . , 9 years and 10 years ahead of a current prediction time) may be used for defining 10 different end points of a sliding time window covering 20 years. Thus, 10 different sub-sets of the retrieved documents may be used as data basis for feature extraction and for generating 10 different, offset-dependent feature sets. Each of the feature sets is fed into a respective classifier for generating an offset-dependent prediction if the drug directed at a particular target will be capable of treating the disease. For example, the first classifier (corresponding to an offset time of 10 years) may output an indication whether the drug directed at said target can treat the disease. For example, said indication can be a binary “yes” or “no” value or can be a likelihood percentage value. For example, said indication can be a likelihood of 49% that the drug directed at said target can treat the disease. The second classifier (corresponding to an offset time of 9 years) may output a likelihood of 53% that the drug directed at the target can treat the disease, and so on. After each of the 10 classifier has output its decision result in the form of a likelihood percentage value, e.g. the median of the 10 likelihood percentage values is computed and output as the final, combined result. The combined result indicates a combined prediction result of whether the outcome of the medical study will be that the drug directed at said target is capable of treating the disease. Instead of the median, the arithmetic mean or other mathematical approach for computing an average or mean value may be used for computing the combined result from the results output by the plurality of classifiers. In case the individual classifiers generate binary prediction results, the combined result can also be a binary result that is identical to the binary result output by the majority of the classifiers.

This may increase the accuracy of the prediction as the combined result integrates information contained in the result generated by multiple classifiers corresponding to multiple different time intervals ahead of the publication of the result of a medical study.

According to embodiments, the time window comprises a plurality of time intervals. For example, the time intervals can be a sequence of consecutive time intervals, typically years.

According to embodiments, the extraction of a plurality of features from the ones of the received documents published during the time window comprises:
assigning each of the received documents to the one of the time intervals that covers the publication day of the document;
for each of the time intervals, extracting a plurality of first features from the ones of the received documents published during said time interval and extracting a plurality of second features from the ones of the received documents published in said and all its preceding time intervals in the window.

Extracting both first features covering only one, comparatively short time interval, e.g. one year, and second features covering a comparatively long time period (typically multiple years) may be advantageous as this kind of feature extraction may be more robust against outliers: in particular in the early years of a new research area, the number of publications per year are small. By computing also cumulative features covering multiple time intervals, the effect of outliers and of the high variability of feature values may be reduced. By computing the first features selectively from documents published in a single interval in addition to the second (cumulative) features, it may be easier to identify trends in feature development over multiple years as the publications of previous years have no impact on the first features extracted for a, single evaluated time interval. Thus, embodiments of the invention provide for a feature extraction approach that is robust against outliers and capable of capturing trends in feature development at the same time.

Thus, the first features may be described as features extracted from documents published in a particular time interval within the window, e.g. within a particular year. A second feature may be described as a feature extracted from documents published within said single year or published in any year preceding said single year and being covered by the time window. According to some embodiments, if in a particular time interval no document was published, the first features computed for said particular time interval is set to zero and the second features computed for said particular time interval are identical to the second features extracted for the time interval directly preceding said particular time interval.

According to embodiments, the windows used for extracting the features for different offset times have the same size. For example, the time window used for extracting feature sets for different time offsets may always cover 20 years. According to embodiments, the time intervals are consecutive time intervals of predefined duration, e.g. of one year duration. The number of consecutive time intervals in a window can be, for example, in the range of 5 to 25, e.g. 20.

To give a concrete example, the window used for extracting the input features of multiple classifiers may always cover the same length, e.g. 20 years. For extracting input features for a classifier trained on a training time offset of “1” years, the window is “shifted” such that it has the time offset of “1” years. This means that the window starts 21 years ahead of the time of prediction and ends at the offset time (one year) ahead of the moment of performing the prediction (i.e., the offset time ahead of the current day). For extracting input features for a classifier trained on a training time offset of “3” years, the window is “shifted” such that it also has the time offset of “3” years, meaning that the window starts 23 years ahead of the time of prediction and ends 3 years ahead of the time of prediction. For extracting input features for a classifier trained on a training time offset of “10” years, the window is “shifted” such that it also has the time offset of “10” years, meaning that the window starts 30 years ahead of the time of prediction and ends 10 years ahead of the time of prediction. Thus, for 10 different offset times 1 year, . . . , 10 years, 10 different window positions are defined, 10 different feature sets are extracted from different sub-sets of biomedical documents, and each of the 10 different feature sets is provided as input to a respective one of 10 trained classifiers, whereby the 10 classifiers were trained on training features having been extracted by the same “sliding window” technique and by using said 10 different offset times.

For example, a particular classifier corresponds to the training offset time of “3” years. The classifier is trained by defining a time window that is to be used for extracting the training features, the time window starting 23 years and ending 3 years ahead of a (known) moment in time when the outcome of a corresponding training study was disclosed.

According to embodiments, each of the predefined, different offset times comprises a consecutive number of years ahead of the moment of performing the prediction. Each of the corresponding predefined, different training offsets respectively comprises a consecutive number of years ahead of the moment the outcome of a training study related to a training target-disease-pair was disclosed. For example, the predefined offset times and the corresponding, predefined training offset times may be in the range of 0 to 15 years. According to one example where 10 different offset times and corresponding training offset times are defined, the first offset time and corresponding training offset time may be “1 year”, a second offset time and corresponding training offset time may be “2 years”, . . . , and the last predefined offset time and corresponding training offset time may be “10 years”.

According to embodiments, the method comprises:
identifying of a publication day of the one of the received documents being the first published document comprising an identifier of either the target or of the disease;
the extraction of plurality of the training features for the specified time window comprising assigning zero values to all features to be extracted for any one of the plurality of time intervals chronologically preceding the time interval comprising said identified publication day. In embodiments where first and second features are extracted, the assignment of zero values can be performed upon extracting the first as well as upon extracting the second features.

According to embodiments, the window covers one or more of the following time intervals:
a time during which basic research on the target and/or the disease is performed; and/or
a time during which target discovery for the disease is performed; and/or
a time during which pre-clinical trials for the drug directed at the target and the disease are performed; and/or
a time during which clinical trials for the drug directed at the target and the disease are performed.

Said features may allow to systematically analyze publication patterns emerging along the drug discovery process (e.g. of targeted cancer therapies), starting from basic research on a particular target to drug approval—or failure. Clear differences in the patterns of approved drugs directed at a particular target compared to those that failed in phase 2/3 of clinical studies were observed regarding several features, whereby the types of features having the greatest predictive power are implemented in various embodiments described herein for extracting test features (i.e., features for performing a prediction) and for extracting training features (i.e., features extracted from training documents and used as input for training a classifier).

According to embodiments, the method comprises automatically querying one or more biomedical databases, for automatically retrieving one or features to be used as a further input to the classifier. For example, the biomedical database can be a protein database like PDB and may comprise information on the location of the target within a cell. For example, the following features may be retrieved from the one or more biomedical databases, e.g., via a network:
data indicating whether the target is expressed on the surface of a cell;
data indicating the level of differential expression in a disease;
structural data of the target allowing a detecting suitable drug binding sites on said target;
the functional class of the target (i.e. “tyrosine kinase”);
structural data of the target allowing the detection of structurally similar targets (e.g. a 3D model of the target); and/or
data being indicative of a biochemical pathway comprising or being influenced by the target.

Said additional features are used as further training features for training a classifier and/or for as further features to be provided as input to a classifier for performing the prediction.

Retrieving additional data on a target from a protein database or other databases and using the data as additional test and training features may be advantageous as said additional features may allow increasing prediction accuracy.

According to embodiments, the extracted features comprise:
“disease-document features”: features extracted selectively from documents comprising an identifier of the disease irrespective of whether said documents comprise an identifier of the target;
“target-document features”: features extracted selectively from documents comprising an identifier of the target irrespective of whether said documents comprise an identifier of the disease; and
“co-occurrence-document features”: features extracted selectively from documents comprising an identifier of the disease and of the target.

Extracting a particular feature type, e.g. “commitment”, from different (e.g. three different) sub-sets of documents (listed above) may be advantageous as it was observed that the accuracy of the classifier was increased.

According to embodiments, the totality of biomedical document comprising an identifier of either the target or of the disease is retrieved via a network from a document source database by an application program and stored on a local storage medium or device. The retrieved documents are re-used multiple times for extracting feature sets for multiple different windows corresponding to multiple different predefined time offsets. Thereby, the first features having been extracted for a particular time interval may be stored to the storage medium and may be re-used when computing the first feature of a time interval of another window if said other window also covers said time interval.

For example, the first window to be specified may be a window w-01 having an offset time of one year, and for a particular first feature type, 20 first features may be computed, one for each time interval of the first window. In a second step, the window is shifted one time interval to the past such that the time offset is two years. Thereby, a new window w-02 is defined having 19 time intervals in common with window w-01. The first features having already been computed for said 19 time intervals that are covered by the first window w-01 and by the second window w-02 are not re-computed but are rather read from the storage medium. Only for the single time interval covered by the second window w-02 but not by the first window w-01, a corresponding additional first feature is computed. This approach may significantly increase performance since at least a part of the features, in particular of the first features, are extracted from the documents only once and are used as input for multiple different classifiers corresponding to different offset times, whereby only the relative position of the time interval from which the first feature was derived differs for different offset times and corresponding windows. At least some of the second, cumulative features are not computed directly by analyzing the documents published during a set of time intervals but are computed by analyzing the first features extracted from documents published during said set of time intervals. This may further increase performance.

According to embodiments, each first feature is provided as input to the classifier in association with an indication of the position of the time interval from which it was retrieved. Analogously, each first training feature is provided as input to the untrained classifier in association with an indication of the position of the time interval from which it was retrieved.

Extracting many different feature types from different subsets of documents may be beneficial, because the analyzer may enabled to perform a feature analysis and prediction on a very rich feature set. These features may allow generating a machine learning classifier capable of predicting the approval or denial of novel drugs directed at a particular target several years in advance.

For example, the first features extracted for each of the different offset times may comprise a mixture of one or more disease-document features, target-document features and co-occurrence-document features. In addition, or alternatively, the second features extracted for each of the different offset times may comprise a mixture of one or more disease-document features, target-document features and co-occurrence-document features.

According to embodiments, any type of feature described herein and having been extracted for providing input data to an already trained classifier corresponds to a respective training feature of identical type extracted from training documents in the same way. Analogously, any type of training feature described herein and having been extracted for providing input data for training a classifier corresponds to a respective feature of identical type extracted from documents in the same way for being provided as input to an already trained classifier.

According to embodiments, the documents are received from a source document database. The extracted features comprise:
a normalized document count; the normalized document count is indicative of the number of documents comprising an identifier of the target and of the disease and being published in the one or more of the time intervals for which the features are extracted, whereby said number of documents are normalized over the totality of biomedical documents published in said one or more time intervals and comprising an identifier of the target or the disease or both; and/or
a commitment index; the commitment index is indicative of the number of authors having published at least two documents comprising an identifier of both the disease and of the target; extracting the “commitment” or “commitment index” feature may be advantageous as said feature indicates the trust of scientific experts into the future therapeutic potential of a research topic; Commitment has been observed to be continuously higher in positive target-disease pairs than in negative target-disease pairs; and/or
“therapeutic MeSH count”: said feature type indicates the number of documents comprising an identifier of the target and/or of the disease and comprising the MeSH major subheadings “drug therapy” and “therapeutic use”.

It has been observed that the above mentioned feature types show the highest predictive power of all examined features. Thus, by extracting features corresponding to one or more of the above three feature types, a high prediction accuracy may be achieved.

For example, the first features extracted for each of the different offset times may comprise a combination of the normalized document count, the commitment index and the “therapeutic MeSH count”. In addition, or alternatively, the second features extracted for each of the different offset times may comprise a combination of the normalized document count, the commitment index and the “therapeutic MeSH count”. Of course, per definition of “first” and “second” features, said three feature types, when computed as “second features” (cumulative features), are computed from a different set of documents as the same type of features when computed as (interval specific) “first features”.

According to embodiments, each of the feature types “normalized document count”, “commitment index” and the “therapeutic MeSH count” are computed as a first feature and in addition as a second feature by using different documents as input for feature extraction. In addition, or alternatively, each of the feature types normalized document count, the commitment index and the “therapeutic MeSH count” are computed as “disease-document feature”, “target-document feature” and “co-occurrence-document feature” by using different documents as input for feature extraction. MeSH (medical subject headings) major subheadings are topic names and annotations assigned by human experts to biomedical documents, e.g. MEDLINE abstracts.

For example, the MEDLINE database can be used as the source document database and the title, abstracts and metadata stored in the MEDLINE database may be used as the biomedical documents.

According to embodiments, the extracted features comprise one or more features selected from a group comprising:
a non-normalized document count, the non-normalized document count being indicative of the number of documents comprising an identifier of the target and/or of the disease;
the numbers of authors of documents comprising an identifier of the target and/or of the disease;
the fraction of authors affiliated to the biotech or pharmaceutical industry, the authors being authors of documents comprising an identifier of the target and/or of the disease and being published in the one or more of the time intervals for which the features are extracted;
the number of genes, chemicals and/or drugs per reference string length which are contained in the documents comprising an identifier of the target and/or of the disease;
the number of occurrences of the phrase “phase 1”, “phase 2” or “phase 3” in the documents comprising an identifier of the target and/or of the disease.

Each or at least some of the above mentioned features are extracted multiple times using different sub-sets of the retrieved documents. For example, for extracting “first features”, a subset of the retrieved documents that is published in a particular year is analyzed while for extracting “second features”, a subset of the retrieved documents that is published in a plurality of consecutive years is analyzed. Only documents covered by the time window or subsets thereof are analyzed for extracting the features.

According to embodiments, each of the above mentioned feature types are computed as a first feature and in addition as a second feature by using different documents as input for feature extraction. In addition, or alternatively, each of said feature types are computed as “disease-document feature”, “target-document feature” and “co-occurrence-document feature” by using different documents as input for feature extraction.

According to embodiments, the trained classifier is a random forest classifier. For example, the randomForest package in R (R statistical computing software “http://www.r-project.org”) may be used.

For example, the drug is a small molecule or a biological. According to said or other examples, the disease is a human cancer or human cancer subtype.

According to embodiments, the method further comprises:
computing a normalized Shannon entropy E according to E=MeSH_{#observed}/MeSH_{#max}, whereby MeSH_{#observed} is the number of MeSH (“Medical Subject Headings”) major subheadings of the retrieved documents, whereby MeSH_{#max} is the number of MeSH major subheadings defined in the MeSH thesaurus, whereby E=0 corresponds to the use of only one MeSH major subheadings in all the retrieved documents and E=1 corresponds to the equal use of all existing MeSH major subheadings; and
using the computed entropy as a measure of the maturity of the biomedical research executed on the target and the disease.

The method may comprise outputting the development of the computed Shannon entropy E for the received documents over the time, e.g. by means of a chart, e.g. a line chart. The chart may indicate the composition of the MeSH major subheadings assigned to the biomedical documents published in a given time interval. Outputting the development of the computed Shannon entropy may be advantageous as this information may allow a human user to determine the maturity of the research relating to the target disease pair.

In a further aspect, the invention relates to a method for training a classifier. The trained classifier is configured to predict an outcome of a medical study. The medical study evaluates the efficacy of a drug directed at a target to treat a disease. The method is implemented in an electronic system and comprises:
providing a set of target-disease training pairs, the set comprising positive target-disease pairs respectively comprising a target whose activity modification is known to treat the disease contained in said target-disease pair, the set further comprising negative target-disease pairs respectively comprising a target whose activity modification is known not to treat the disease contained in said target-disease pair;
specifying a training offset time, the training offset time indicating a time interval ahead of a moment the outcome of a training study related to the target-disease training pairs was disclosed, each training study designed to evaluate the efficacy of a drug directed at the target to treat the disease specified in the target-disease training pair;
specifying a time window of predefined duration, the window ending at the training offset time;
for each of the target-disease training pairs of the set:
   receiving biomedical training documents comprising an identifier of the target or of the disease or of the target and the disease of the target-disease training pair;
   extracting a plurality of training features selectively from the ones of the received documents published during said time window;
generating the trained classifier by training an untrained classifier selectively on the training features extracted for the target-disease training pairs for the specified training offset time.

According to embodiments, the training offset time is one of a plurality of different, predefined training offset times. The method comprises, for each of the predefined training offset times:
specifying a further time window of predefined duration, the window ending at the training offset time;
for each of the target-disease training pairs of the set:
   receiving biomedical training documents comprising an identifier of the target or of the disease or of the target and the disease of the target-disease training pair;
   extracting a plurality of training features selectively from the ones of the received documents published during said further time window;
generating a trained classifier by training the untrained classifier selectively on the extracted training features.

According to embodiments, the time window comprises a plurality of time intervals. The method comprises, for each of the target-disease training pairs:
identifying a publication day of the one of the received training documents being the first published document comprising an identifier of either the target or of the disease of the target-disease training pair;
identifying the one of the plurality of time intervals comprising the identified publication day;
the extraction of plurality of the training features comprising assigning zero values to all training features to be extracted for any one of the plurality of time intervals chronologically preceding the identified one time interval.

For example, if a drug directed at a particular target needed only 15 years till approval, ant the corresponding target-disease pair is used as a training target-disease pair for training a classifier while using a window of 20 years length, the features for years 1-5 are padded with zeros. Thus, it is possible to use the approach for a plurality of different training target-disease pairs, including those where the time period between first publication of a document comprising an identifier of both the disease and of the target and the end of the study is smaller than the time window size.

According to embodiments, the set of target-disease training pairs further comprises a plurality of control target-disease training pairs. A control target-disease pair is a data set comprising a substance not having been used or tested as a target of a drug for treating the disease contained in the target-disease pair.

According to embodiments, the method for training the one or more classifiers according to any one of the embodiments described herein in addition comprises using the generated one or more trained classifiers for performing the method for predicting the efficacy of the drug directed at a target for treating the disease according to any one of embodiments of the prediction method described herein.

In a further aspect, the invention relates to a non-volatile storage medium comprising instructions which, when executed by a processor, cause the processor to perform a method according to any one of the embodiments described herein.

In a further aspect, the invention relates to an electronic system for predicting an outcome of a medical study. The medical study evaluates the efficacy of a target directed at a target to treat a disease. The system comprises a processor configured for:
receiving biomedical documents comprising an identifier of the target or of the disease or of both;
specifying an offset time, the offset time indicating a time interval ahead of the performing of the prediction;
specifying a time window of predefined duration, the window ending at the begin of the offset time;
extracting a plurality of features selectively from the ones of the received documents published during said time window;
providing a classifier having been trained on a set of training features extracted from a set of biomedical training documents, the training documents published within a training time window ending at the begin of the offset time ahead of a moment the outcome of one or more training studies on training target-disease-pairs was disclosed;
performing the prediction by executing the classifier, thereby providing the extracted features as input to the classifier;
outputting a result of the classifier, the result predicting the efficacy of the drug directed at the target to treat the disease.

A “feature” as used herein is a quantitative property extracted from one or more documents or from metadata associated with the one or more documents. A feature extracted directly from one or more documents could be, for example, a feature extracted from the text of the document by applying text mining methods such as named entity recognition, concurrence evaluation, syntactically and/or semantically parsing the text. A feature extracted from metadata of one or more documents could be, for example, a feature extracted by analyzing the author, publication day, type of journal or keywords the document is annotated with.

A “document” as used herein is a set of data wherein the information is provided in a textual form. For example, a document may be a full text article of a biological, biochemical or medical journal, a data record of a biological or medical database, or a part of an electronic article, e.g. an abstract. A document may have assigned meta data such as author, year of publication, keywords (e.g. MeSH terms), links to other documents, etc.

A “classifier” as used herein is a program logic, e.g. a software module or software program, configured for processing input data for performing a prediction, whereby the result of the prediction classifies an object. For example, a classifier may predict that a medical study relating to the efficacy of a drug directed at a target to treat a disease will have the outcome that the drug directed at said target is able to treat the disease. For example, the classifier may predict that the FDA will approve the drug as one or more studies proved the safety of the drug and proved the drug's efficacy in treating the disease. Thereby, the classifier classifies said drug as a substance that is directed at a target whose modification (likely) results in the treatment of a particular disease. Alternatively, the classifier may classify the drug as being a substance that is directed at a target whose modification will (probably) be incapable of treating the disease.

A “target” or “drug target” as used herein is a defined molecule or structure within the organism, typically a protein, that is linked to a particular disease, and whose activity can be modified by a drug, whereby the modification of the activity of the target is a mechanism for treating the disease.

A “time window” as used herein is a bounded time interval which is characterized by a starting time and an end time, whereby the end time is specified by an offset time relative to a particular moment in time. Said “particular moment in time” can be, for example, a time when a prediction is performed, e.g. when input data is provided to a classifier to execute the classifier on the input data. In a training phase of a classifier, the end time of the time window used for selecting the documents from which the training features are to be extracted is specified by a training offset time relative to a particular moment in time when the outcome of a training medical study was published, thereby revealing whether a modification of an activity of a particular target of a training target-disease pair was capable of treating said disease or not.

A “drug” or “medicine” as used herein is any substance other than food, that when inhaled, injected, smoked, consumed, absorbed via a patch on the skin or dissolved under the tongue causes a physiological change in the body. A drug is typically used to treat, cure, prevent, diagnose a disease or promote well-being by modifying the activity of a drug target. Drugs may be used for a limited duration, or on a regular basis for chronic disorders.

A “disease” as used herein is an abnormal condition, a disorder of a structure or function that affects part or all of an organism. It may be caused by factors originally from an external source, such as infectious disease, or it may be caused by internal dysfunctions, such as autoimmune diseases or cancer. A disease as used herein may also refer to a particular form of a disease, e.g. a particular form of a cancer such as breast cancer or lung cancer characterized by a particular biomarker expression pattern.

A “medical study” as used herein is a scientific examination of how a drug directed at a particular target and applied as a treatment for disease works in a group of organisms, e.g. a group of patients or laboratory animals. A medical study can be, for example, a study performed in the context of a research project for doing basic research on the biochemical effects of a substance, can be performed as a pre-clinical study and/or can be performed as a clinical study of the first, second or third phase. A medical study can be, for example, a study performed for obtaining an approval of the FDA for a particular drug, and the day when the outcome of a study is disclosed may correspond to the day when the FDA declares if a particular drug will or will not be approved based on the data generated during the study.

A “biological” as used herein is a compound produced by living cells, such as proteins, enzymes and amino acids. A “small molecule” as used herein is a low molecular weight (<900 daltons) organic compound that helps regulating or is suspected to regulate a biological process.

A “target-disease pair” as used herein is a combination, represented e.g. in the form of a data object, of a particular target and a particular disease. A training target-disease pair is a target-disease pair used with a known biomedical relation between the target and the disease or with a known absence of such a relation, whereby a training target-disease pair is used as part of a training data set for training one or more classifiers.

An “electronic system” as used herein is a data processing system comprising a storage medium and one or more processors for processing data stored in the storage medium. For example, the electronic system can be a standard computer system, a server system or a cloud computer system.

An “identifier” of a disease or a target as used herein is a name or a synonym of said disease or of said target.

### BRIEF DESCRIPTION OF THE DRAWINGS

In the following embodiments of the invention are explained in greater detail, by way of example only, making reference to the drawings in which:

FIG. 1 is a line chart depicting a growing number of publications for a target-disease pair;

FIG. 2 is a block diagram of a system configured for training one or more classifiers and/or for using the one or more trained classifier for predicting the efficacy of a drug directed at a particular target;

FIG. 3 depicts Venn diagrams of subsets of the retrieved documents;

FIGS. 4A-C depict trends for different features extracted from documents related to targeted anti-cancer drugs before FDA approval or failure;

FIG. 4D depicts the F-measure of the prediction;

FIG. 5 depicts features extracted for three different classes of target-disease pairs;

FIG. 6 depicts a flow chart of a prediction method according to an embodiment;

FIG. 7a depicts a publication trend for a target-disease pair before FDA approval;

FIG. 7b depicts a time window having an offset time of 5 years;

FIG. 8 depicts time windows having offset times of 2 and 3 years;

FIG. 9 depicts a chart illustrating a change in the distribution of MESH major subheadings over the time;

FIG. 10 depicts time dependency of the F-measure of three different types of classifiers; and

FIG. 11 depicts trends of features extracted from biomedical documents retrieved for three different target-disease pairs.

### DETAILED DESCRIPTION

FIG. 1 is a line chart 100 depicting a growing number of publications in the scientific literature for a target-disease pair in the field of targeted cancer therapies. The x-axis represents a time scale covering 20 years and the y axis indicates the number of publications per year comprising an identifier of both the target and of the disease of a given target-disease pair. The first appearance of biomedical documents, e.g. scientific articles describing a target molecule in the context of and together with a particular disease, e.g. a particular cancer type, is followed by a stream of “continuous research” on this subject. Moreover, the pharmaceutical R & D process starts which may comprise the following phases: target identification/validation (TI/V) for identifying the target whose activity modification can treat a disease, identification of a lead compound (IL) (the process of identifying a drug or drug version that is particularly suitable or effective for modifying the activity of the target), lead optimization (LO) (the process of optimizing a potential drug that shall modify the activity of the target), pre-clinical tests (PC), phase 1, 2 and 3 clinical trials (P1, P2, P3) and approval and launch (AL) for a particular drug directed at the target for treating the disease. Thus, basic research and pharmaceutical R&D generate signals in the literature by publishing on various aspects of the target in the context of a particular disease (that may also be referred to as “indication”).

At the end of the medical study, the drug may be approved by a government authority, such as the United States Food and Drug Administration (FDA) or the authority may issue a decision not to approve the drug for treating the disease. In addition or alternatively, at the end of a medical study the result may be published in a scientific journal.

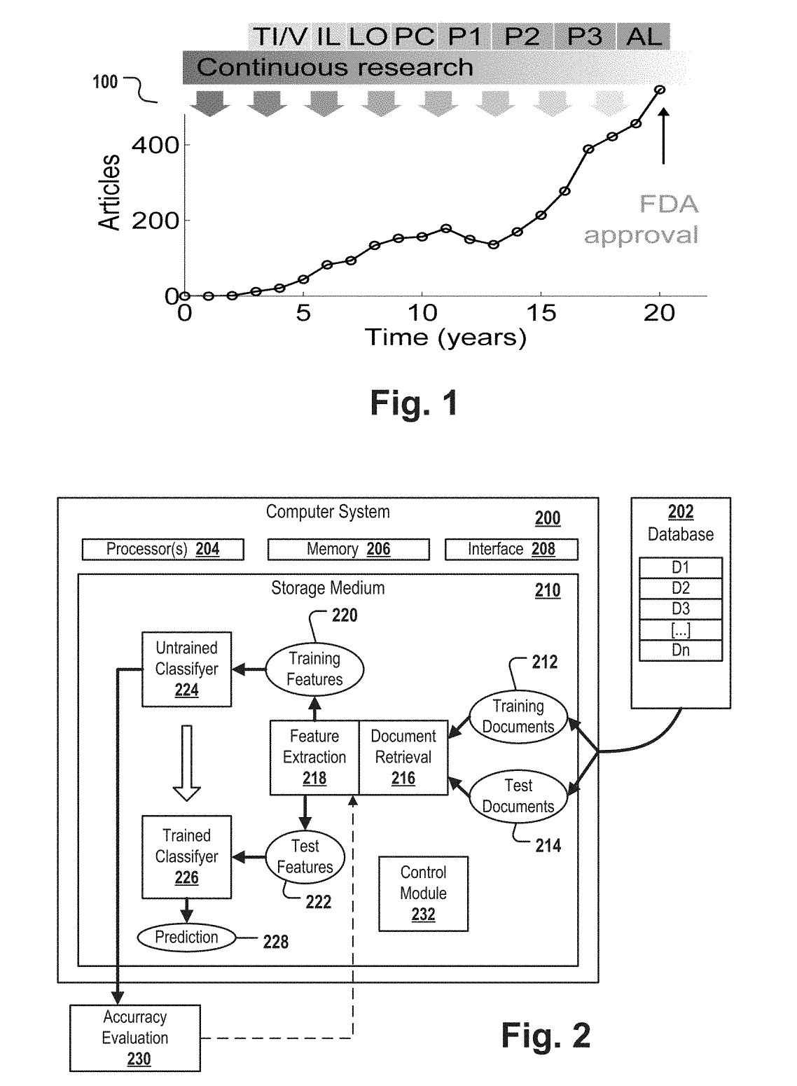

FIG. 2 is a block diagram of a system configured for training one or more classifiers and/or for using the one or more trained classifier for predicting the efficacy of a drug directed at a particular target for treating a disease. The system comprises one or more pieces of program logic configured for performing a method as described, for example, in FIG. 6. In the following, reference will be made to FIG. 2 and FIG. 6.

The electronic system 200 comprises or is operatively coupled to a database 202 comprising a plurality of biomedical documents D1, D2, . . . , Dn. For example, the database 202 may be a local copy of the MEDLINE database comprising more than 24 million biomedical abstracts. The computer system comprises one or more processors 204, a main memory 206, a non-volatile storage medium 210 and an interface 208 for enabling a user to control and/or inspect the process of training one or more classifiers and/or the process of using the one or more classifiers for predicting the outcome of a medical study. The electronic system may be, for example, a computer system, e.g. a server or standard desktop PC. The system comprises one or more program modules 216, 218, 226, 230 configured for predicting an outcome of a medical study and/or for generating one or more machine-learning based classifiers from untrained classifiers 224. The medical study evaluates the efficacy of a drug directed at a target to treat a disease. The whole process may be coordinated and controlled by a control module 232 and operating with the document retrieval module 216, the feature extraction module 218, some further modules for training and untrained classifier, for sampling the training data sets and for generating and outputting a result of the prediction generated by one of the classifiers 228.

In a first step 602, a document retrieval module 216 receives a plurality of biomedical documents 214. The plurality of received documents comprise a) documents comprising an identifier of the target or b) an identifier of the disease or c) identifiers of the target and of the disease. The retrieved documents can be stored as a subset for later processing in a different table in the database 202 or as a file in the non-volatile storage medium 210.

In a further step 604, the control module 232 and/or a user specify an offset time. The offset time indicates a time interval ahead of the time of performing of the prediction. For example, in case all steps 602 to 614 depicted in FIG. 6 are executed on a particular day, said particular day is the “time of prediction”. In some embodiments, at least some of the features used as input in the prediction may be extracted earlier and the time of performing step 612 is used as the time of performing the prediction. Preferentially, multiple different offset times are defined. For example a set of 10 different offset times may be defined: 1 year ahead of the prediction, 2 years ahead of the prediction, . . . , 9 years ahead of the prediction and 10 years ahead of the prediction.

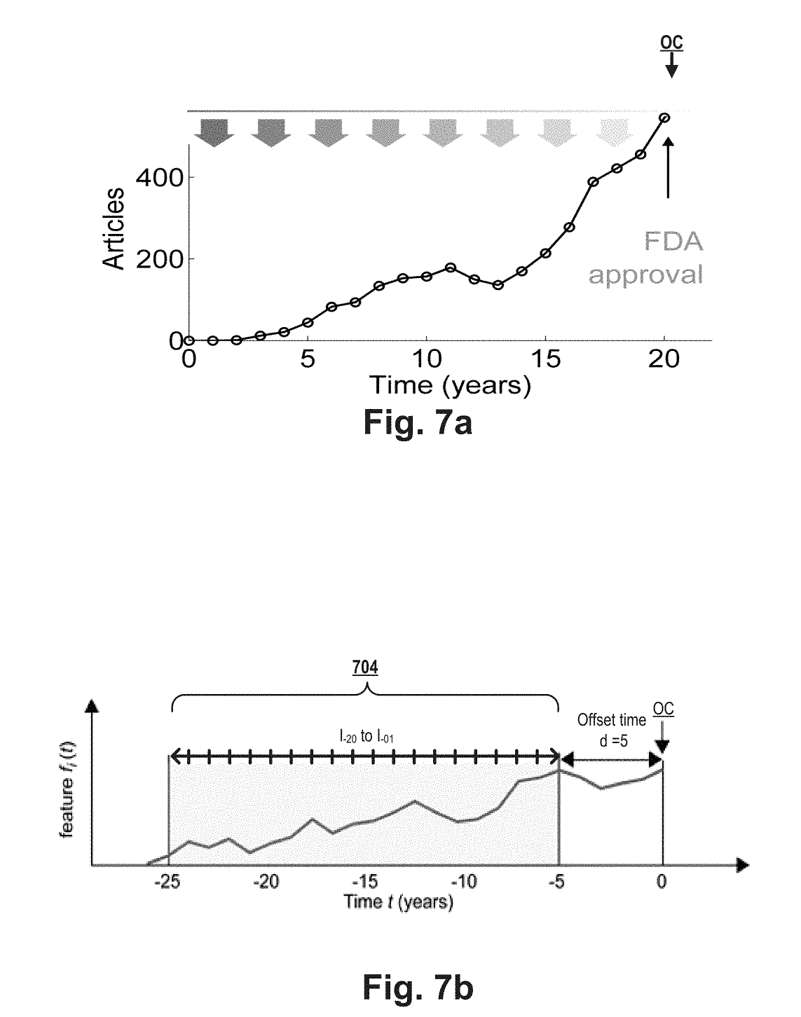

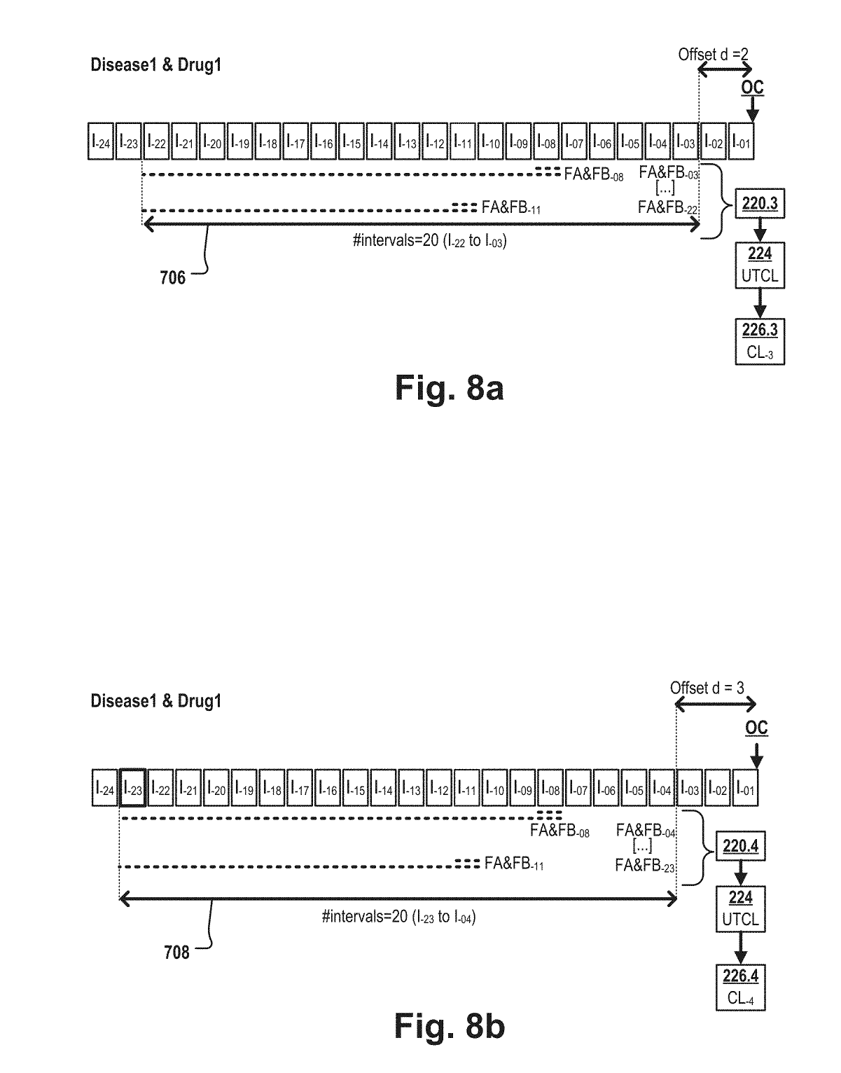

In a further step 606, the control module 232 and/or a user specifies a time window of predefined duration, e.g. 20 years. The time window ends at the begin of the offset time. For each of the offset times, a respective time window can be defined. FIGS. 7b, 8a and 8b show different time windows 704, 706 and 708.

In a further step 608, the control module extracts a plurality of features 222 (in distinction to the training features 220 being also referred to as “test features”) selectively from the ones of the received documents published during said time window. This step is repeated for each of the time windows having been defined in step 606, thereby respectively using different subsets of the received documents as input and extracting different sets of features (whereby at least the features extracted on an per-time-interval basis can be shared by multiple ones of said feature sets).

Step 610 comprises providing a classifier 226.3 having been trained on a set of training features 220.3. The training features have been extracted from a set of biomedical training documents which were published within a training time window ending at the begin of the offset time ahead of a moment OC the outcome of one or more training studies on training target-disease-pairs was disclosed. For each of the defined windows and corresponding test features, a respective classifier is provided having been trained on a respective set of training features. For example, for a window whose offset time is 3 years ahead of the prediction time in step 612, a classifier 226.3 is retrieved having been trained on training features 220.3 which were extracted from a set of training documents published within a time window of the same size and having an offset time of 3 years before the result of a study with known outcome (“training study”) was disclosed. For a window whose offset time is 4 years ahead of the prediction time in step 612, a classifier 226.4 is retrieved having been trained on training features 220.4 which were extracted from a set of training documents published within a time window of the same size and having an offset time of 4 years before the result of a study with known outcome was disclosed (see FIGS. 8a and 8b). Thus, for 10 different windows, 10 respective trained classifiers may be provided.

In step 612, each of the provided classifiers is executed, thereby using a corresponding set of extracted features 222.3, 224 as input of the classifier. The classifier performs, based on the input features 222, a prediction of the efficacy of the drug directed at the target to treat the disease. A feature set “corresponding” to a classifier is a test feature set having been extracted from documents published during a time window whose width and time offset is identical to a training time window used for identifying the documents from which the training features used for training said classifier were extracted.

In step 614, each of the executed classifiers outputs a respective result 228, the result predicting the efficacy of the drug directed at the target to treat the disease.

Finally, in case multiple classifiers 226.1, . . . , 226.10 (one per defined time window) were executed sequentially or in parallel, the results output by the plurality of executed classifiers are combined by the control module for generating a combined result. For example, the first classifier may compute a likelihood that the outcome of the medical study is that the drug directed at the target can be used to treat the disease of 71%. The second classifier may compute a likelihood of 83%. The third classifier may compute a likelihood of 76% and so on up to the 10th classifier. The combined likelihood can be computed, for example, as the median or mean likelihood of all the likelihoods computed by the individual classifiers. Alternatively, the output of each classifier may be a binary “yes” or “no” prediction whether the outcome of the medical study will be that the drug is effective in treating the disease (and optionally, is in addition safe) or not. The final combined result of all classifiers may be computed by performing a voting process, and the final combined result may be identical to the binary “yes” or “no” prediction output by the majority of the classifiers.

Optionally, the system may comprise an accuracy evaluation module 230 that automatically evaluates the accuracy of the trained classifiers on a training data set comprising training documents and training target-disease pairs. The results obtained by the accuracy evaluation module can be used for determining the impact of individual features on the prediction accuracy of a classifier and the predictive power of said feature.

The above steps have been described for a situation where already one or more trained classifiers 226 exist which are applied on input features 222 having been extracted from a set of documents 224 (“test documents”) defined by a currently used window.

The training phase for generating the trained classifiers from an untrained version 224 of the classifier is performed analogously: a plurality of training target-disease pairs is defined whereby at least for some of said pairs, the positive or negative outcome of a medical study (referred herein as training study) is known. The windows used in the training phase (“training time windows”) are defined using an offset time that is defined relative to and ahead of the day when the outcome of the study is disclosed. For each training target-disease pair a set of documents is retrieved mentioning the target or the disease of the training target-disease pair or mentioning both. Each training time window respectively defines a subset of the received documents used for extracting a set of training features. The training features extracted for a particular offset time and for a plurality of documents retrieved for a plurality of different training target-disease pairs in combination with information on the outcome of the training studies is input to an untrained classifier for generating a trained classifier being specific for said offset time.

In the following, a concrete example for generating a set of trained classifiers and for using the trained classifiers for predicting the efficacy of a drug directed against a particular target for treating a particular disease will be given.

### Defining a Training Data Set Comprising Multiple Classes of T-DI Pairs

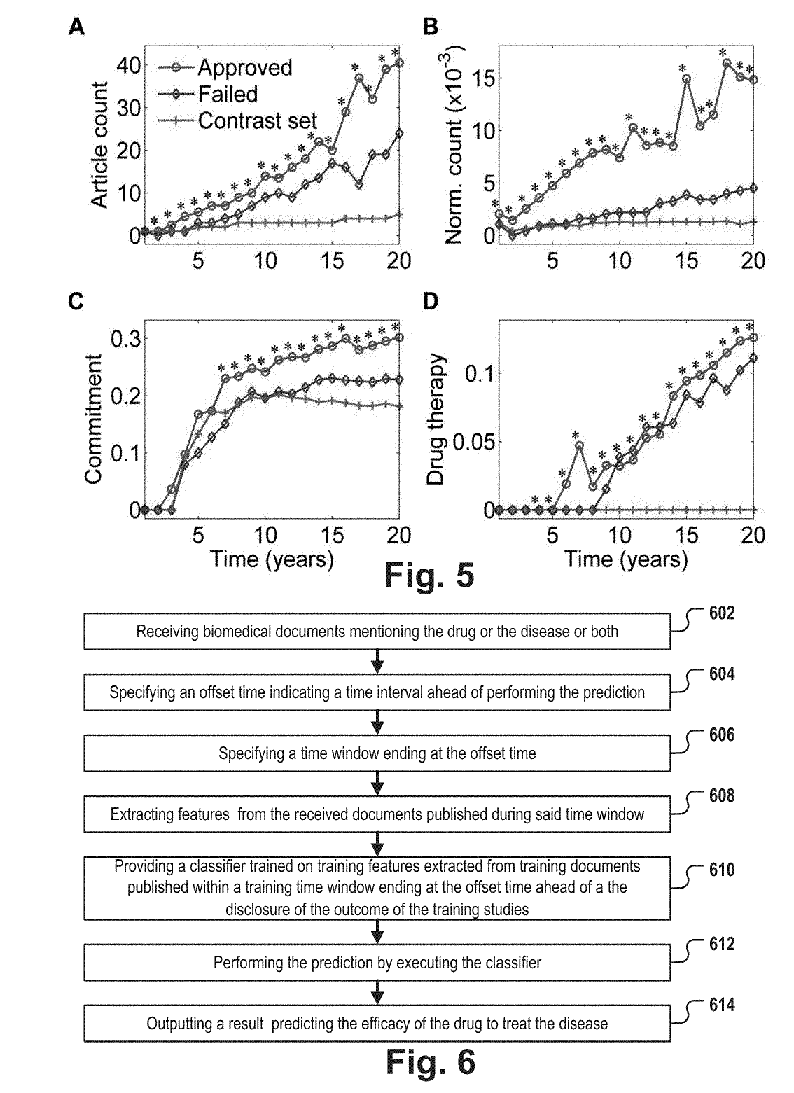

In analogy to the classes of T-DI pairs described in FIG. 5, at least two classes of target-disease pairs were collected: (1) Target-disease pairs corresponding to approved targeted anti-cancer drugs, and (2) target-disease pairs corresponding to targeted anti-cancer drugs failed in phase 2/3 clinical trials. Optionally, a third class (3) of target-disease pairs can be compiled which do not correspond to any targeted anti-cancer drug that has either been approved or tested in a clinical trial of phase 1 or later.

More precisely, class 1 contains target (T)-disease (DI) pairs, where T is a target for a successfully approved anti-cancer drug against disease DI. To obtain these T-DI pairs, a list of FDA approved targeted anti-cancer drugs was generated, using data from the national cancer institute (NCI) website (www.cancer.gov) and the US food and drug administration (FDA) website (www.fda.gov) retrieved in September 2014. A list of all targets T for the approved drugs and related diseases DI was generated. The drugs of the T-DI pairs comprised small molecules and biological. For these T-DI pairs, the FDA approval year was stored in a T-DI matrix containing the class 1 cases. For example, for target “ERBB2” and disease “Breast cancer” the approval year is “1998” (the FDA approval year of the ERBB2 (Her2) targeting drug Trastuzumab (Roche, Basel, Switzerland)). In case of multiple drug approvals for a T-DI combination, the earliest approval year was used as the “decision time” OC. In case of multiple targets (T₁, T₂, . . . ) for a given disease were known, the target with the highest publication count of said T-DI pairs was used. Drugs with unknown targets or more than three targets were excluded in accordance with the procedure of Joshi and Milletti (Joshi, V. and Milletti, F. (2014) “Quantifying the probability of clinical trial success from scientific articles”, Drug discovery today 19 (10), 1514-1517).

42 unique, positive target-disease training pairs containing FDA approved targeted drugs and respective diseases were obtained. In addition, 74 negative target-disease training pairs related to targeted anti-cancer drugs which failed in phase 2/3 clinical trials were obtained.

To find failed phase 2 or 3 clinical trials, the Pharmaprojects and TrialTrove (Citeline, Informa, London, UK) databases and the U. S. National Institutes of Health clinical studies registry (www.clinicaltrials.gov) were used. The search was conducted in December 2014. Failure of a drug targeting T as a treatment for DI was defined by the trial outcomes “Terminated, lack of efficacy”, “Terminated, Safety/adverse effects” or “Completed, negative outcome/primary endpoint not met”. In case of drug combinations, only new targeted drugs were considered, which are not yet approved as treatment for the respective disease (i.e., if drug 1 is approved in a combination with a previously approved drug 2 as treatment for disease DI, only the target T of the new drug 1 is considered). If a non-successful trial was found, the year of failure along with the classification of each T-DI pair was stored. In case of multiple failed trials, the earliest year was taken.

Class 3 represents a contrasting set of T-DI pairs which do not correspond to any targeted anti-cancer drug and have not been in clinical trials or already been approved. The T-DI pairs were determined using the same diseases as used in class 1 and 2 of the T-DI pairs. The proteins acting as target T were obtained from the human protein atlas project (http://www.proteinatlas.org). Here, the subset of cancer-related proteins without those labeled as FDA approved drug targets was selected (“protein_class:Cancer-related genes NOT protein_class:FDA approved drug targets”). The subset was retrieved in February 2015. The set of cancer-related genes in the human protein atlas is a combination of data from the Plasma Proteome Institute, comprehensive published catalogues of cancer specific genes and the catalogue of somatic mutations in human cancer (COSMIC, cancer.sanger.ac.uk). From this set of 1555 proteins, 50 proteins were randomly selected as targets and combined with a plurality of different diseases to form a third class of T-DI pairs, also referred to as “control group of T-DI pairs”. The control group comprised 299 T-DI pairs. A manual verification was performed to ensure that none of these 50 proteins had been used as a drug target in a clinical trial.

### Retrieval of Training Documents for a Plurality of T-DI Pairs

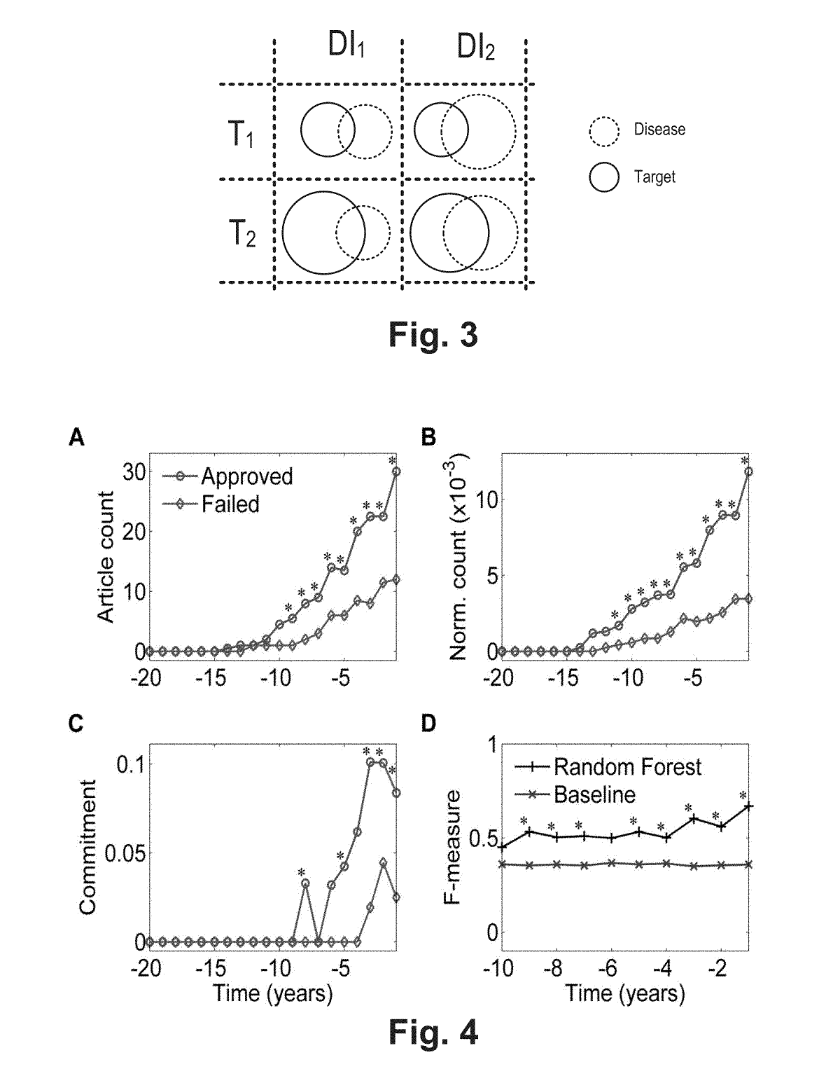

At first, names and synonyms for the diseases and targets of the training disease-target pairs were retrieved by combining terms derived from multiple data sources comprising Entrez Gene, Uniprot and Panther. For the diseases, a terminology combining MeSH terms and the NCI thesaurus was used for extracting disease names and their synonyms. Terms empirically known to result in false positives, for example terms which are also acronyms in another context, were removed from the list of synonyms. The output of each query is a text file with rows consisting of hits for the search terms used, i.e., a target name and synonyms thereof or a disease name and synonyms thereof. The Venn diagrams of FIG. 3 illustrate that the set of documents retrieved for a particular target may be used for feature extraction for multiple different target-disease pairs. This may increase performance as it is not necessary to retrieve the same set of documents multiple times for the different T-DI pairs in case e.g. two or more T-DI pairs share the same target or the same disease.

For each training T-DI pair from classes 1 and 2, and optionally also from class 3 (control), related scientific literature was retrieved from MEDLINE. For this purpose, the MEDLINE corpus (in total ˜23 10⁶ publications, state September 2014) was processed with the text mining platform I2E enterprise (Linguamatics, Cambridge, United Kingdom) to find documents mentioning at least one identifier (name or synonym) of the target and/or of disease of each of the training T-DI pairs. For each target and for each disease, a single query was executed and a single result file was generated. The search for the respective entities of T or DI was restricted to title and abstract respectively constituting a “document” in this example approach. Then, the documents comprising an identifier of the disease and comprising an identifier of the target of each training T-DI pair were obtained by computing the intersection of the PubMed IDs in the publication result files respectively retrieved for the target and the disease of each pair.

### Meta Data Processing and Enrichment

Each document comprises meta data. The meta data comprises, for example, the publication year, the PubMed id and the major MeSH subheadings. In addition, the metadata was automatically supplemented with a string containing a company name by analyzing the author names of a document and performing a lookup in a database comprising known affiliations of biomedical scientists with a pharmaceutical or biotech company. In addition, genes and chemicals were identified in the documents and meta data of said genes and chemicals was retrieved from further data sources such as GeneView for enriching the metadata of the documents with biomedical information related to the genes and chemical substances mentioned therein.

### Feature Extraction

The retrieved documents and their respectively (and optionally supplemented) metadata is then used to compute features fᵢ(t) for a predefined set of feature types, where i denotes the i-th feature type, where t denotes a “relative time” corresponding to a predefined set of relative times The features are computed for each of a predefined set of offset times d and could therefore likewise be denoted as f_{di}(t) where d denotes the offset time from which the relative time t depends.

For the comparison of the positive and negative training T-DI pairs (i.e., T-DI pairs of class 1 and class 2), the relative time t was computed relative to the respective “decision time” OC (the time when the outcome “OC” of a study was disclosed, e.g. drug approval or failure of clinical trials). A plurality of predefined offset times d (d∈{1 . . . 10} years) is used for computing a set of relative times t i.e., t=y−OC, where y is the year of the publication and OC is the time of the decision event.

For each of the computed relative times t and for each of a plurality of pre-defined feature types i, a feature fᵢ(t) was computed from documents published in or prior to a year covering the relative time t, where i-denotes the i-th feature at the relative time t.

Preferentially, the positive and negative training T-DI pairs are chosen such that the average time span ranging from the first document with a co-occurrence of T and DI to the approval or failure decision time OC shows no significant difference for the positive and negative training T-DI pairs. This eliminates the possibility of a high temporal offset of one class.

In the current case, the following findings for the different T-DI classes were obtained: class 1 median time span: 15.5 years, 25th and 75th percentiles: 10.25 and 22 years; n=42; class 2: median time span: 16 years, 25th and 75th percentiles: 10.25 and 16 years; n=74. No significant difference for both T-DI classes were observed (p<0.05, Mann-Whitney-Wilcoxon test, two-tailed).

Moreover, the positive and negative training T-DI pairs are chosen such that the absolute year of the decision (disclosing the outcome of the study) does not differ significantly for the positive and negative training T-DI pairs. This may reduce a potential bias for the case that the underlying patterns change with time.

In the current case, the following findings for the different T-DI classes were obtained: class 1 publication year median: 2009, 25th and 75th percentiles: 2004 and 2012; n=42; class 2 publication year median: 2008, 25th and 75th percentiles: 2006 and 2010; n=74. No significant difference for both T-DI classes were observed (p<0.05, Mann-Whitney-Wilcoxon test, two-tailed).

In addition, the T-DI pairs of the “control” T-DI pair class 3 were compared against the T-DI pairs of classes 1 and 2. The time after the first publication of a document mentioning both the target and the disease of a given T-DI pair was analyzed forward in time and the relative time t was determined according to t=y−y₀, with y being the year of the publication of the document and y₀ the year of said first publication.

For all T-DI classes the time window investigated was 20 years (i.e., 20 years prior to approval or failure for the comparison of class 1 and 2 and 20 years after the first publication for the analysis of class 3 compared to class 1 and 2 respectively).

If there were no publications for a T-DI pair in a given year, values for cumulative, “second” features (e.g. the cumulative publication count) were set to the value of the first previous year with a publication, while feature values of non-cumulative (“first”) features (e.g. the publication count in a specific year) were set to zero. If the time span ranging from the year of the first publication to the approval or failure of a T-DI pair in class 1 or class 2 was less than 20 years, the feature data was padded with zeros for the feature values in the years prior to the first publication, such that all time windows have a length of precisely 20 years.

The training features derived for the T-DI pairs of class 1 vs. class 2 were used as training set for generating sets of classifiers using several machine learning approaches, namely naïve Bayes, decision trees, random forests, support vector machines and binary logistic regression. To find characteristic features depending on the offset time (“distance”) d to the time OC of disclosing approval or failure of a drug, 10 different classifiers were trained using the features of extracted from documents published during a time window of 20 years, whereby the time window was shifted for different values of an offset time d (d∈{1 . . . 10} years) before the decision time OC. Data contained in documents published during the d years before the decision time was omitted. The time window of 20 years comprises a sequence of time intervals I of predefined length, e.g. is a sequence of 20 time intervals respectively covering one year (see FIG. 8). Each of said time interval corresponds to a respective relative time t.

More formally, for the specific T-DI pairs with known approval or failure at decision time OC, and after conversion to relative times t (relative to and ahead of the decision time), the feature values fᵢ(t) for a plurality of different relative times t corresponding to respective time intervals I was computed as t=Δt−w−d with Δt∈{1, . . . , w}, where w is the number of time intervals within the time window, were used to train the d-th classifier (see FIG. 8). For a time window covering 20 years and comprising 20 “one-year” time intervals, the relative times at which a feature is extracted is t=Δt−20−d with Δt∈{1, . . . , 20}.

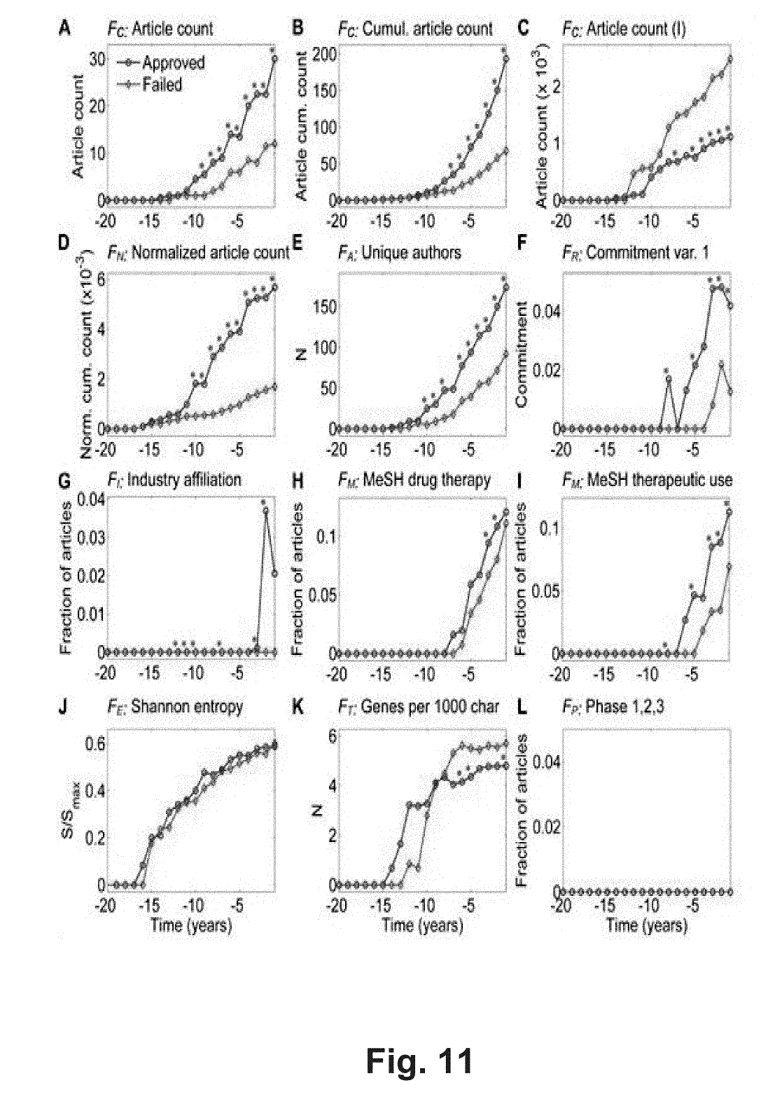

FIG. 10 depicts time dependency of the F-measure of three different types of classifiers predicting the approval of a targeted drug: (B) Random forest classifier. (C) Decision tree classifier. (D) Support vector machine (SVM) classifier. As a baseline, the F-measure obtained by guessing using the known a-priori distribution of the training examples is shown. Asterisks indicate a significant difference (p<0.05, Welch's t test, two-tailed). Error bars represent the standard error of the mean. It has been observed that the random forest classifier shows the highest accuracy. This is a surprising observation as random classifiers have been observed not to be accurate (Hastie, Trevor; Tibshirani, Robert; Friedman, Jerome 2008 “The Elements of Statistical Learning”, 2nd ed., Springer, ISBN 0-387-95284-5, p. 352).

### Features

In the following, a plurality of features are described that have been observed to have a sufficient, good or even high predictive power in respect to the question if a particular drug directed at a particular target is able to treat a disease or not. The features belong to different feature classes. Each feature class is a set of one or more realizations of the features. In the following feature classes and features are listed. The superscript of the feature symbol corresponds to the feature class:
The feature subscript ‘TDI’ corresponds to features (“co-occurrence-document features”) obtained using publications from a T-DI document set (i.e., a sub-set of the retrieved documents mentioning both the target and the disease);
Subscripts ‘T’ corresponds to features (“target-document features”) extracted from documents mentioning at least the targets (irrespective of the disease);
Subscripts ‘DI’ corresponds to features (“disease-document features”) extracted from documents mentioning at least the disease (irrespective of the targets) were used.
Subscript ‘y’ denotes a feature extracted only from documents published during one year and thus represents a “first feature”. In FIG. 8, the first features are denoted as “FA”.
Subscript ‘c’ denotes a cumulative feature, also referred herein as “second feature”, and is computed by extracting data from retrieved documents whose publication day lies within a time window and lies before or in the year comprising the relative time t for which the feature is computed, and by summing up the extracted data. In FIG. 8, the second features are denoted as “FB”.

If not denoted as a feature subscript, the T-DI document set was used and the feature computed per year (“first feature”).
1. Feature class “Article counts” or “document counts”: F_{C}
   Features: f^{C}_{TD1y}, f^{C}_{TDIc}, f^{C}_{TDIc}, f^{C}_{Ty}, f^{C}_{Tc}, f^{C}_{DIy}, f^{C}_{DIc}
   The number n1 of documents (per year and cumulative) comprising an identifier of the disease and of the target of a T-DI pair is determined as a feature according to n1 (T, DI)=|T∩DI|.
   In addition, the number n2 of documents mentioning at least the target n2(T)=|T| (irrespective of the occurrence of a disease identifier) is extracted as a feature.
   Moreover, the number n3 of documents mentioning at least the disease (irrespective of the occurrence of a target identifier) n3(DI)=|DI| is extracted as feature.
2. Feature class “Normalized document (article) counts”: F_{N}
   Features: f^{N}_{TDIy}, f^{N}_{TDIc}, f^{N}_{Ty}, f^{N}_{Tc}, f^{N}_{DIy}, f^{N}_{DIc}
   The number of documents n1=|T∩DI| normalized by the total number n4 of documents n4=|T∪DI| for the union of documents comprising an identifier of the target or of the disease:

$$n 4 ( T , D I ) = \vert \frac{T ⋂ DI}{T ⋃ DI} \vert .$$

3. Feature class “Authors”: F_{A}
   Features: f^{A}_{ay}, f^{A}_{ac}, f^{A}_{uy}, f^{A}_{uc}, f^{A}_{dy}, f^{A}_{dc}, f^{A}_{nc}
   Encompasses features measuring the absolute number of authors (feature subscript ‘a’), unique authors (feature subscript ‘u’), authors with more than one publication (feature subscript ‘d’) and the average number of authors per paper (feature subscript ‘n’).
4. Feature class “Research commitment”: F_{R}
   Features: f^{R}_{1y}, f^{R}_{1c}, f^{R}_{2y}, f^{R}_{2c}
   An heuristic for the number of people actively doing research on a target-disease combination, approximated by the fraction of authors who published more than one article about it.
   Variant 1:

$${c}_{1} = \frac{\vert R \vert}{\vert A \vert}$$

with A the set of all authors for a specific T-DI combination and R the subset of authors with more than one document mentioning both the disease and the target (feature subscript 1).
Variant 2:

$${c}_{2} = {\sum }_{r ∈ R} f ( r ) \text{/} {\sum }_{a ∈ A} f ( a )$$

with f(x) the number of publications of an author x in the respective sets A or R (feature subscript 2).
5. Feature class “Industry affiliation”: F_{I}
   Feature: f^{I}
   The fraction of documents comprising an identifier of at least one pharmaceutical or biotech company in the meta data of the document.
6. Feature class “MeSH subheadings”: F_{M}
   Feature: f^{M}s
   The distribution of major MeSH subheadings (also referred to as qualifiers). MeSH has a total of 83 subheadings (numeric feature subscript s, s∈{1 . . . 83}) which are used to describe specific aspects of MeSH terms used.
7. Feature class “Normalized Shannon entropy of MeSH qualifiers”: F_{E}
   Feature: f^{E}
   Normalized Shannon entropy to quantify the heterogeneity of used MeSH terms. The Shannon entropy S=−Σᵢ₌₁^{N}pᵢ log₂ pᵢ of the frequencies of the N=83 major MeSH subheadings (pᵢ=1/nᵢ, with nᵢ representing the number of times the i-th subheading was found in a set of documents) normalized by the Shannon entropy Sₘₐₓ for the case of equal probability of all subheadings (p=1/N) S/Sₘₐₓ ∈{0 . . . 1}. In the current case, Sₘₐₓ is 83 but this number may differ in dependence on the thesaurus used for computing the entropy. S/Sₘₐₓ=1 represents a perfectly homogeneous distribution of subheadings (i.e., documents with a very wide distribution of topics) and S/Sₘₐₓ=0 or very small represents a very heterogeneous distribution of subheadings (i.e., all documents have an identical topic).
8. Feature class “Biomedical terms count”: F_{T}
   Features: f^{T}ₕ, f^{T}_{d}, f^{T}_{g}
   The number of chemicals (feature subscript h), drugs (subscript d) and genes (subscript g) mentioned in a document (e.g. the abstract of a publication) relative to a reference string length, e.g. relative to a 1000 character word string.
9. Feature class “Phase term count”: F_{P}
   Features: f^{P}ₚ₁, f^{P}ₚ₂, f^{P}ₚ₃
   The number of documents mentioning either “phase 1”, “phase 2” or “phase 3” (and synonyms), normalized to the total number of documents for a T-DI pair (feature subscripts p1, p2, p3).

Any one of the above mentioned features can be used, alone or in combination with other features, as training features for training one or more classifiers and/or as test features for predicting the outcome of a clinical study for determining if a particular disease can be treated by a drug directed at a particular target.

These comparisons led to a number of interesting findings depicted in FIG. 4. FIG. 4A shows that starting at nine years before an FDA approval, the class of approved T-DI pairs shows a significantly increased document count compared to eventually failing pairs. Differences are even more pronounced for an even larger temporal distance to approval/failure when using a normalized document count which takes into account a-priori frequencies of targets and diseases (FIG. 4B). FIG. 4C shows that the commitment score of approved drugs, which measures the number of times individual authors publish on a T-DI pair, is significantly higher than that of failed drugs, with the difference becoming significant at three consecutive years before FDA approval. Equally interesting patterns show up when analyzing the distribution of MeSH major subheadings over time. In particular, subheadings “drug therapy” and “therapeutic use” are annotated considerable more frequently to papers mentioning successful targeted drugs than for those on non-approved drugs (FIG. 5D).

Also other features differ significantly between both classes (FIG. 11). Typically, these differences become clearly visible a few years before approval or failure, such as in the case of industry affiliations (FIG. 11G) or counts of gene mentions (FIG. 11K). Features based on data for a specific year are more often significantly different than their cumulative counterparts (e.g. FIG. 11A, B). This is due to the fact that the accumulation of information mixes signals significant in some time spans with non-significant signals from other time spans. In addition, potential differences in the publication patterns between small molecule drugs and biologics were analyzed by separately analyzing both drug classes. Both exhibited similar trends in features and thereby legitimate their combined analysis.

According to one example, to predict a drug approval in d years, features from an interval of 20 years until d years before OC were extracted (FIG. 7, 8). A separate classifier for each offset time d was trained and evaluated using 10-fold cross validation. A clear trend towards better classification performance at shorter distances d for two of the classification methods (random forest and decision tree, FIG. 10) was observed. These classifiers performed much better than a baseline which guesses outcome based on the a-priori distribution of successes and failures in the training data.

The best observed machine learning approach was using a random forest classifier as described for example in Breiman, L. (2001): “Random forests”, Machine learning 45 (1), 5-32 whose disclosure is included herewith by reference in its entiredy. Compared to the F-measure of the baseline (F≈0.36), this classifier performed significantly better already 5 consecutive years before the formal decision on the drug fate. The F-measure starts at F=0.45±0.08 (mean±standard error of mean) at 10 years ahead of time (accuracy, A=0.58±0.06) and increases to F=0.67±0.05 one year before the decision (A=0.73±0.04).

By extracting a combination of features comprising at least a normalized publication count, commitment, and occurrence of MeSH terms “drug therapy” and “therapeutic use” a particularly high prediction accuracy may be achieved with low computational effort during feature extraction.

FIG. 3 depicts Venn diagrams of document sets retrieved for four different target-disease pairs (T1-DI1), (T1-DI2), (T2-DI1), (T2-DI2), wherein T1 represents a first target, DI1 represents a first disease, T2-represents a second target and D12 represents a second disease. Documents comprising an identifier of the target and the disease of a particular target-disease-pair were identified by retrieving documents comprising at least an identifier for a target (T), by retrieving documents comprising at least an identifier of the diseases (DI) and intersecting them to find publications with T-DI co-occurrences.

FIGS. 4A-C depict trends for different features extracted from biomedical documents which are related to targeted anti-cancer drugs before FDA approval or failure in phase 2 or 3. The moment in time when the outcome of a medical study is disclosed (e.g. a decision of the FDA to approve the drug for being used to treat a particular disease or a decision to refuse said approval) is positioned at time t=0.

Median annual feature values for up to 20 years before this event (i.e. disclosure of the outcome of a study) are shown. Asterisks next to the features indicate significant differences (p<0.05, Mann-Whitney-Wilcoxon test, two-tailed) of respective feature values between approved and non-approved drugs.

The following features are depicted in FIG. 4: (A) document count (“article count”) per year, i.e., the number of documents published per year which mention the disease and the target (“co-occurrence-document feature”). (B) normalized document count per year, i.e., the feature of (A) normalized by the total number of biomedical documents of the document source published during said year. For example, the name of a particular disease D1 may be mentioned in 1.300 documents published in a reference time, e.g. in a particular year. The total number of documents published during this reference time may be 1 Mio. So the normalized document count would be 1.300/1 Mio; (C) Commitment per year, a feature being indicative of the number of authors having published at least two papers comprising the identifier of the target as well as the disease per year. Said feature captures the tendency of authors to publish multiple papers on a given disease-target pair.

Feature (A) mentioned above is a co-occurrence-document feature. Optionally, for one or more of said features, also a “disease-document feature” (the number of documents published per year which mention the disease irrespective of whether said documents also mention the target) and/or a “target-document feature” (the number of documents published per year which mention the target irrespective of whether said documents also mention the disease) can be computed.

FIG. 4D depicts an F-measure of multiple different random forest classifiers respectively having been trained on different training feature sets respectively derived by using a different offset time. Each classifier predicts drug approval or failure at varying distances (“offset times”) to the “decision time” (when the outcome of the study is disclosed) at time t=0). The time-independent baseline indicates an estimated outcome that is computed based on the a-priori ratio of approvals/failures in the training data used for training the classifier(s). Asterisks indicate a significant difference (p<0.05, Welch's t test, two-tailed) of the accuracy of a classifier's prediction of the outcome of a study compared with a random guess based on said a-priori ratio. The accuracy of the prediction can be increased by combining the prediction result generated by each of the classifiers for generating a combined result.

FIGS. 5A-5D depict various features extracted from biomedical documents comprising target identifiers and disease identifiers of target-disease pairs. The features can be used as training features. The features depicted in FIG. 5A-5C correspond to the features described for FIG. 4A-4C.

The documents from which the features are extracted are training documents compiled for three different classes of target-disease pairs as follows: a list of targeted drugs that were either approved by the FDA as a treatment against an oncological disease (class “Approved”, n=42) or failed in phase 2/3 clinical trials (class “Failed”, n=74) was compiled.

In other words, a first class (“Approved”) of T-DI pairs comprises “positive” target-disease pairs respectively comprising a target whose activity modification was experimentally verified (“is known”) to treat the disease contained in said target-disease pair. The second class (“Failed”) comprises negative target-disease pairs respectively comprising a target whose activity modification was experimentally verified (“is known”) not to be capable of treating the disease contained in said target-disease pair. The third class (“control class” or “contrast set”) comprises target-disease pairs whose target is a substance not having been used or tested as a target of a drug for treating the disease contained in said pair.

The corresponding drug targets (T) and diseases (DI) were used to form T-DI pairs and related documents were retrieved from MEDLINE using text mining. Preferentially, all documents mentioning (“comprising a name or a synonym”) the drug or mentioning the disease or mentioning the disease and the drug were retrieved. Next, features were extracted from the received documents (in this case: MEDLINE abstracts) and their metadata. Multiple different types of features were extracted and analyzed, encompassing simple features such as document count, author count, counts of said retrieved documents in addition comprising an identifier of genes, chemicals, or drugs, or plain occurrences of the terms “phase 1/2/3” are determined.

Additionally, the number of authors who are actively doing research on a specific T-DI combination (commitment) as well as the fraction of authors affiliated to the pharmaceutical/biotech industry were determined. Both feature types may indicate trust of scientific experts into the future therapeutic potential of a research topic. Moreover, the distribution of MeSH major subheadings, i.e., topics describing the document content annotated by human experts, was analyzed and a subset of specific MeSH major subheadings were identified whose occurrence is a good predictor of drug approval.

Each T-DI pair was associated with a specific point-in-time, the decision time (OC) also referred herein as the time when a result of a study for determining the efficacy of a drug directed at a particular target to tread a disease is disclosed. For T-DI pairs of approved drugs, OC is the year of FDA approval. For failed drugs, OC is the year of the trial failure. For each T-DI pair, yearly features were computed and plotted using a time window ranging from t=−1 to t=−20 years before the OC (t=0) and compared the medians of features for approved drugs to those of failed drugs.

Features were extracted from documents retrieved for positive target-disease pairs for which an approved drug exists (class 1), for negative target-disease pairs for which a “failed drugs” exists (class 2) and, optionally, for the contrast set (class 3).

The document analysis for extracting the features starts with the first document comprising an identifier of the disease and an identifier of the target of said target-disease pair and being contained the currently used time window. Thus, the moment t=0 indicating the start of the analysis depicted in FIG. 5 is a different time than the time tin FIG. 4 which is defined by the moment of performing the prediction.

FIGS. 5A-D depicts median annual feature values, whereby the median is computed from multiple features of the same type derived from multiple target-disease pairs of the same class. The depicted features are: (A) document count per year. (B) document count per year, normalized by the total number of documents published in said year (including those neither mentioning the disease nor the target). (C) Commitment per year. (D) Fraction of the number of documents retrieved for a particular target-disease pair and published in a particular year and having assigned the MeSH major subheading “drug therapy” relative to the total number of documents retrieved for said particular target-disease pair and published in said particular year. Asterisks indicate a significant difference between class 1 (approved) and class 3 (contrast set), (p<0.05, Mann-Whitney-Wilcoxon test, two-tailed).

FIG. 7a depicts the growth in the number of documents (“articles”) published within a time period of 20 years before a time OC when a drug directed against a particular target was approved (or finally denied approval) by the FDA for treating a particular disease. The day of approval by the FDA is considered here as the day when the outcome of a medical study for determining whether a particular drug directed at a particular target can be used to treat a disease was disclosed.

FIG. 7b depicts a time window 704 covering 20 years and having an offset time of 5 years before a day OC when a drug directed against a particular target was approved or finally denied approval by the FDA for treating a particular disease. The window comprises 20 time intervals I₋₂₀ to I₋₁ respectively covering 1 year. The features fᵢ(t) were analyzed at relative times t prior to the decision time OC. Machine learning classifiers were trained to predict approval or failure d years in the future, whereby d corresponds to the offset time and the end of the window 702. For each offset time (“distance”) d, different sets of training features were extracted and a different classifier 226.1, . . . , 226.10 was trained (with d=1 . . . 10 years). To ensure an identical data handling for all d classifiers, features from a time window of 20 years were used (gray area) shifted by the distance d (e.g., d=5 in the scheme). Hence features fᵢ(t) were used from the time interval −20+1−d≤t≤−d. More recent data in the range −d+1≤t≤0 were omitted, since it corresponds to unknown future data, when transferred to a current example (i.e., a new T-DI pair—“target-disease pair” with unknown outcome in d years).

FIG. 8a depicts a time window 706 comprising 20 time intervals I₋₂₂ to I₋₀₃ and having an offset time of 3 years. Each of the time intervals covers one year. The time window 706 may be used as a training time window. Extracting test features or training features from a set of documents published during said time window may comprise extracting first and second features for each of the time intervals. For example, for the time interval I₋₀₈, first features FA₋₀₈ are extracted from the ones of the received documents published during said time interval I₋₀₈. In addition, a plurality of second features FB₋₀₈ are extracted from the ones of the received documents published in said time interval I₋₀₈ or published in any of its preceding time intervals I₋₀₉ to I₋₂₂; I in the window 706. For space reasons, only the first FA₋₀₈ and second FB₋₀₈ features of interval I₋₀₈ and the first FA₋₁₁ and second FB₋₁₁ features of interval I₋₁₁ are depicted, but the extraction of the first and second features is performed for each of the time intervals in the window. The totality of first and second features extracted for each time interval of the window 706 is used as input feature set. If the feature extraction is used in a training phase, the extracted features are training features 220.3 that are used as input for an untrained classifier 224 for generating a trained classifier 226.3 for the offset time of 3 years.

FIG. 8b depicts a time window 708 comprising 20 time intervals I₋₂₃ to I₋₀₄ and having an offset time of 4 years. Window 708 can be generated by shifting window 706 one year to the past. Extracting test features or training features from a set of documents published during said time window may comprise extracting first and second features for each of the time interval 708. For example, for the time interval I₋₀₈, first features FA₋₀₈ and second features FB₋₀₈ can be extracted from respective documents as described for FIG. 8a. Alternatively, it is possible that at least the first features FA having already been extracted for windows with different offset times are reused. In the depicted example only the first features for the time interval I₋₂₃ have to be extracted and computed de novo. The second features FB₋₂₃ to FB₋₀₄ are cumulative features gathering information from documents of multiple time intervals preceding a particular time interval for which the features are computed. Thus, the second features may have to be recomputed for each of the time intervals for each of a predefined set of different offset times. If the feature extraction is used in a training phase, the extracted features are training features 220.4 that are used as input for an untrained classifier 224 for generating a trained classifier 226.4 for the offset time of 4 years.

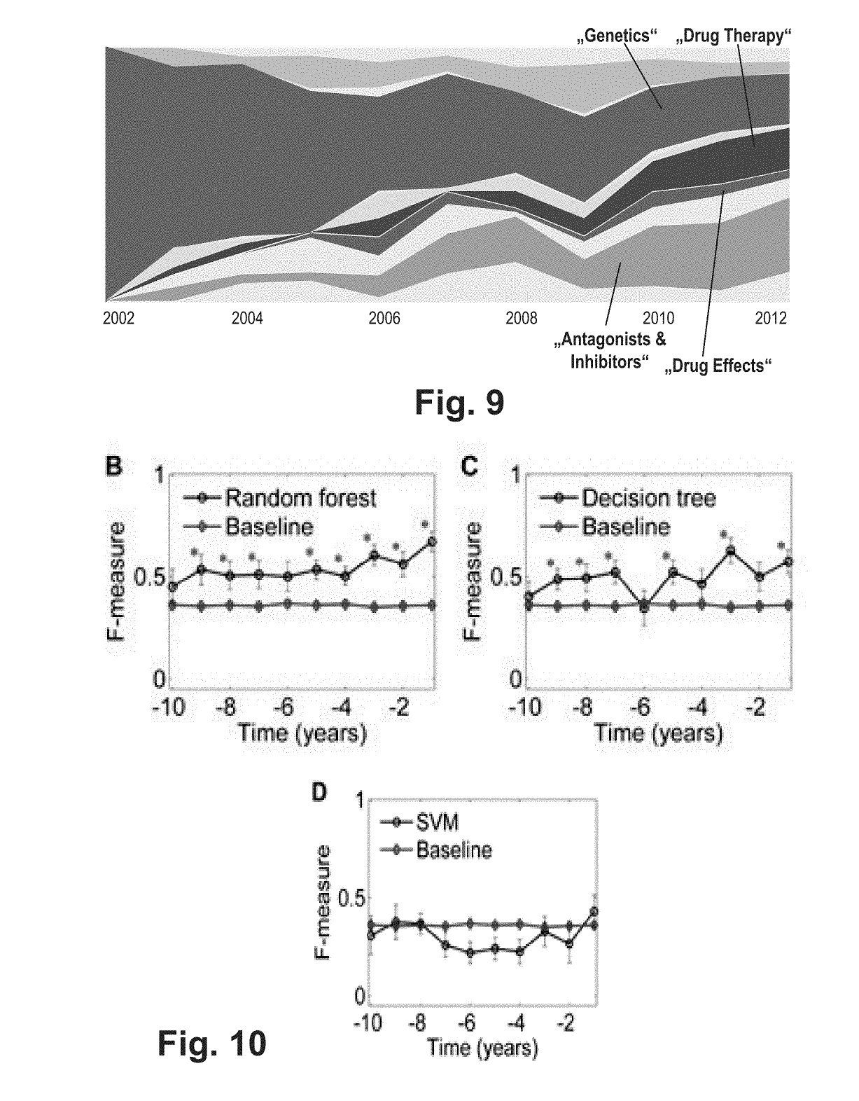

FIG. 9 depicts a chart illustrating a change in the distribution of MESH major subheadings specified in the meta data of the biomedical documents over the time. For each year the MeSH major subheading distribution for documents with co-occurrence of the terms “BRAF” (a target) and “melanoma” (a disease) were determined. This target-disease combination corresponds to the small molecule drug Vemurafenib (Zelboraf®, Roche, Basel, Switzerland). The six most frequent subheadings are indicated by areas of different grey values. In 2005 the compound was available. A subsequent shift in the distribution of topics is visible: The subheadings “drug therapy”, “drug effects” and “antagonists & inhibitors” are annotated more frequently. The more fundamental topic “Genetics” continuously decreases, starting already after the first documents in 2002. In 2011 the drug was approved by the FDA (“decision time”: the outcome of a study was disclosed). In this specific case the subheading “Therapeutic use” was not among the six most frequent subheadings but in general, this feature is a good indicator that a particular target may be a suitable target for treating the disease. The fraction of subheadings is defined by the fraction of documents PA to documents PB, whereby PA is the total set of documents published in a given time window, comprising an identifier of the target, comprising an identifier of the disease and containing the respective subheadings whereby PB is the total set of documents published in a given time window, comprising an identifier of the target, comprising an identifier of the disease.

The MeSH major subject headings whose development over time is depicted in FIG. 9 may be used for computing the feature “normalized Shannon entropy of MeSH major subheadings” (f^{E}) as described herein for embodiments of the invention. The increase in entropy (“disorder”) is also graphically derivable from FIG. 9.

According to embodiments, the Shannon entropy for different years is plotted and displayed on a display device. This may be beneficial as the user is provided with a visual indication of the maturity of a research area which again may assist a user in assessing the maturity a particular field has reached at the moment of performing the prediction. As prediction accuracy is higher for mature fields of research, this may assist a user in assessing the accuracy of the current prediction.

FIG. 11 depicts various features extracted from documents received for three different classes of target-disease pairs. The drugs are targeted anti-cancer drugs having been approved (class 1) or rejected in phase 2 or 3 (class 2) by the FDA.

The respective decision time “OC” (approval or failure) is located at OC=0 and feature medians up to 20 years before the decision time are shown. At least one selected feature from each of the nine feature classes is shown. The respective feature class is indicated by the two letter abbreviation above each plot. (A) Document (“Article”) count per year (f^{C}_{TDIy}). (B) Cumulative document (“article”) count (f^{C}_{TDIc}). (C) Document count for diseases per year (f^{C}_{DIy}). (D) Normalized document count (f^{N}_{TDIc}). (E) Number of unique author names per year (f^{A}_{uy}). (F) Author commitment per year (f^{R}_{1y}). (G) Fraction of documents per year with affiliated pharmaceutical or biotech company (f_{DI}). (H) Fraction of documents per year using the MeSH major subheadings “drug therapy” (I) and “therapeutic use” (f^{M}ₛ). (J) Normalized Shannon entropy of the MeSH major subheadings with S/Sₘₐₓ=0 corresponding to the use of only one subheading and S/Sₘₐₓ=1 to the equal use of all MeSH subheadings (f^{E}). (K) Number of genes (per 1000 characters) mentioned in the document per year (f^{T}_{g}). (L) Fraction of documents published per year mentioning either “phase 1”, “phase 2”, “phase 3” (f^{P}_{p1,2,3}) or synonyms thereof. Asterisks next to the feature values indicate a significant difference (p<0.05, Mann-Whitney-Wilcoxon test, two-tailed).

1. A method for predicting an outcome of a medical study evaluating the efficacy of a drug directed at a target to treat a disease, the method being implemented in an electronic system and comprising:
receiving biomedical documents comprising an identifier of the target or an identifier of the disease or identifiers of the target and of the disease;
specifying an offset time (d), the offset time indicating a time interval ahead of the performing of the prediction;
specifying a time window of predefined duration, the window ending at the begin of the offset time;
extracting a plurality of features selectively from the ones of the received documents published during said time window;
providing a classifier having been trained on a set of training features extracted from a set of biomedical training documents, the training documents published within a training time window ending at the begin of the offset time ahead of a moment the outcome of one or more training studies on training target-disease-pairs was disclosed;
performing the prediction by executing the classifier, thereby providing the extracted features as input to the classifier;
outputting a result of the classifier, the result predicting the efficacy of the drug directed at the target to treat the disease.

2. The method of claim 1, the offset time being one of a plurality of different, predefined offset times, the trained classifier being one of a plurality of trained classifiers having been trained on training features extracted from biomedical training documents published within a training time window, the training time windows of each of the classifiers ending at a different training offset time ahead of a moment the outcome of one or more training studies on training target-disease-pairs was disclosed, the method comprising, for each of the predefined offset times:
specifying a further time window of predefined duration, the further window ending at the predefined offset time;
extracting a plurality of features selectively from the ones of the received documents published during said further time window;
providing the extracted plurality of features as input selectively to the one of the plurality of classifiers having been trained on a set of training features extracted from training documents published within a training time window ending at a training offset time that is identical to the predefined offset time;
performing the prediction by executing the classifier to which the features were provided; and
outputting a result of the classifier, the result predicting the efficacy of the drug directed at the target to treat the disease.

3. The method of claim 2, further comprising:
combining the results output by the plurality of executed classifiers for generating a combined result, the combined result being indicative of whether the outcome of the medical study will be that the drug directed at the target can be used to treat the disease.

4. The method of claim 1, the time window comprising a plurality of time intervals.

5. The method of claim 4, the extraction of a plurality of features from the ones of the received documents published during the time window comprising:
assigning each of the received documents to the one of the time intervals that covers the publication day of the document;
for each of the time intervals, extracting a plurality of first features from the ones of the received documents published during said time interval and extracting a plurality of second features from the ones of the received documents published in said and all its preceding time intervals in the window.

6. The method of claim 4, the time intervals being years, the number of time intervals within the time window being in the range of 5 to 25.

7. The method of claim 2, the predefined offset times comprising a consecutive number of years ahead of the moment of performing the prediction, the training offset times comprising a consecutive number of years ahead of the moment the outcome of the one or more training studies on training target-disease pairs was disclosed.

8. The method of claim 4, further comprising:
identifying of a publication day of the one of the received documents being the first published document comprising an identifier of either the target or of the disease;
the extraction of plurality of the training features for the specified time window comprising assigning zero values to all features to be extracted for any one of the plurality of time intervals chronologically preceding the time interval comprising said identified publication day.

9. The method of claim 1, the time window covering:
a time during which basic research on the target and/or the disease is performed; and/or
a time during which target discovery for the disease is performed; and/or
a time during which pre-clinical trials for the drug directed at the target and the disease are performed; and/or
a time during which clinical trials for the drug directed at the target and the disease are performed.

10. The method of claim 1, further comprising:
automatically querying one or more biomedical databases for automatically retrieving additional features, the additional features being selected from a group comprising:
data indicating the location of the target within a cell;
data indicating whether the target is expressed on the surface of a cell;
data indicating the level of differential expression in a disease;
structural data of the target allowing a detecting suitable drug binding sites on said target;
the functional class of the target;
structural data of the target allowing the detection of structurally similar targets; and/or
data being indicative of a biochemical pathway comprising or being influenced by the target;
and providing the additionally retrieved features as a further input to the classifier.

11. The method of claim 1, the features comprising:
features extracted selectively from documents comprising an identifier of the disease irrespective of whether said documents comprise an identifier of the target;
features extracted selectively from documents comprising an identifier of the target irrespective of whether said documents comprise an identifier of the disease; and
features extracted selectively from documents comprising an identifier of the disease and of the target.

12. The method of claim 1, the documents being received from a source document database, the extracted features comprising:
a normalized document count, the normalized document count being indicative of the number of documents comprising an identifier of the target and of the disease and being published in the one or more of the time intervals for which the features are extracted, the number of documents being normalized over the totality of biomedical documents published in said one or more time intervals and comprising an identifier of the target or of the disease or of both; and/or
a commitment index, the commitment index being indicative of the number of authors having published at least two documents comprising an identifier of the disease and of the target; and/or
number of documents comprising an identifier of the target and/or of the disease and comprising the MeSH major subheadings “drug therapy” and “therapeutic use”.

13. The method of claim 1, the extracted features comprising one or more features being selected from a group comprising:
a non-normalized document count, the non-normalized document count being indicative of the number of documents comprising an identifier of the target and of the disease;
the numbers of authors of documents comprising an identifier of the target and/or of the disease;
the fraction of authors affiliated to the biotech or pharmaceutical industry, the authors being authors of documents comprising an identifier of the target and/or of the disease;
the number of genes, chemicals and/or drugs per reference string length which are contained in the documents comprising an identifier of the target and/or of the disease;
the number of documents comprising at least one of the phrases “phase 1”, “phase 2” or “phase 3” or a synonym thereof, the documents in addition comprising an identifier of the target and/or of the disease.

14. The method of claim 1, the trained classifier being a random forest classifier.

15. The method of claim 1, the drug being a small molecule or a biological and/or the disease being a human cancer or human cancer subtype.

16. The method of claim 1, further comprising:
computing a normalized Shannon entropy E according to E=MeSH_{#observed}/MeSH_{#max}, whereby MeSH_{#observed} is the number of MeSH major subheadings of the retrieved documents, whereby MeSH_{#max} is the number of MeSH major subheadings defined in the MeSH thesaurus, whereby E=0 corresponds to the use of only one MeSH major subheadings in all the retrieved documents and E=1 corresponds to the equal use of all existing MeSH major subheadings; and
using the computed entropy as a measure of the maturity of the biomedical research executed on the target and the disease.

17. A method for training a classifier, the trained classifier being configured to predict an outcome of a medical study, the medical study evaluating the efficacy of a drug directed at a target to treat a disease, the method being implemented in an electronic system and comprising:
providing a set of target-disease training pairs, the set comprising positive target-disease pairs respectively comprising a target whose activity modification is known to treat the disease contained in said target-disease pair, the set further comprising negative target-disease pairs respectively comprising a target whose activity modification is known not to treat the disease contained in said target-disease pair;
specifying a training offset time, the training offset time indicating a time interval ahead of a moment the outcome of a training study related to the target-disease training pairs was disclosed, each training study designed to evaluate the efficacy of a drug directed at the target to treat the disease specified in the target-disease training pair;
specifying a time window of predefined duration, the window ending at the training offset time;
for each of the target-disease training pairs of the set:
receiving biomedical training documents comprising an identifier of the target or of the disease or the target and the disease of the target-disease training pair;
extracting a plurality of training features selectively from the ones of the received documents published during said time window;
generating the trained classifier by training an untrained classifier selectively on the training features extracted for the target-disease training pairs for the specified training offset time.

18. The method of claim 17, the training offset time being one of a plurality of different, predefined training offset times, the method comprising, for each of the predefined training offset times:
specifying a further time window of predefined duration, the window ending at the training offset time;
for each of the target-disease training pairs of the set:
receiving biomedical training documents comprising an identifier of the target or of the disease or the target and the disease of the target-disease training pair;
extracting a plurality of training features selectively from the ones of the received documents published during said further time window;
generating a trained classifier by training the untrained classifier selectively on the extracted training features.

19. The method of claim 17, the time window comprising a plurality of time intervals, the method comprising, for each of the target-disease training pairs:
identifying a publication day of the one of the received training documents being the first published document comprising an identifier of either the target or of the disease of the target-disease training pair;
identifying the one of the plurality of time intervals comprising the identified publication day;
the extraction of plurality of the training features comprising assigning zero values to all training features to be extracted for any one of the plurality of time intervals chronologically preceding the identified one time interval.

20. (canceled)

21. A non-transitory storage medium comprising instructions which, when executed by a processor, cause the processor to perform a method according to claim 17.

22. An electronic system for predicting an outcome of a medical study, the medical study evaluating the efficacy of a drug directed at a target to treat a disease, the system comprising a processor configured for:
receiving biomedical documents comprising an identifier of the target or an identifier of the disease or identifiers of the target and of the disease;
specifying an offset time, the offset time indicating a time interval ahead of the performing of the prediction;
specifying a time window of predefined duration, the window ending at the begin of the offset time;
extracting a plurality of features selectively from the ones of the received documents published during said time window;
providing a classifier having been trained on a set of training features extracted from a set of biomedical training documents, the training documents published within a training time window ending at the begin of the offset time ahead of a moment the outcome of one or more training studies on training target-disease-pairs was disclosed;
performing the prediction by executing the classifier, thereby providing the extracted features as input to the classifier;
outputting a result of the classifier, the result predicting the efficacy of the drug directed at the target to treat the disease.
